(19) United States
(12) Patent Application Publication
(10) Pub. No.: US 2026/0144984 A1
(43) Pub. Date: May 28, 2026

(54) PROSTHETIC VALVE PACING SYSTEMS

(71) Applicant: SMARTVALVES LTD, Savyon (IL)

(72) Inventors: Yossi GROSS, Moshav Mazor (IL); Navot RABBAN, Ramat Gan (IL); Meni IAMBERGER, Kfar Saba (IL)

(73) Assignee: SMARTVALVES LTD, Savyon (IL)

(21) Appl. No.: 19/409,867

(22) Filed: Dec. 5, 2025

Related U.S. Application Data
(63) Continuation-in-part of application No. PCT/IL2024/050830, filed on Aug. 18, 2024.
Application No. PCT/IL2024/050830 is a continuation-in-part of application No. 18/607,638, filed on Mar. 18, 2024.
Application No. 18/607,638 is a continuation-in-part of application No. 18/452,229, filed on Aug. 18, 2023, now Pat. No. 11,931,255.
Application No. 18/452,229 is a continuation-in-part of application No. 18/452,216, filed on Aug. 18, 2023, now Pat. No. 11,975,203.

(51) Int. Cl. A61N 1/05; A61B 5/287; A61B 5/29; A61F 2/24

(52) U.S. Cl. CPC A61N 1/059; A61B 5/287; A61B 5/29; A61F 2/2418

(57) ABSTRACT

A prosthetic cardiac valve is provided, which is configured to be delivered to a patient in a constrained delivery configuration, and which includes a frame and a plurality of prosthetic leaflets. The frame defines a central longitudinal axis when the prosthetic cardiac valve is in an expanded deployment configuration, and includes interconnected stent struts arranged so as to define interconnected stent cells that include upstream-most stent cells and non-upstream-most stent cells. The plurality of prosthetic leaflets are coupled to the frame so as to allow blood flow in a downstream direction and inhibit blood flow in an upstream direction. The prosthetic cardiac valve further includes first and second electrodes, which are disposed at or near respective upstream peaks of first and second ones of the non-upstream-most stent cells, respectively. Other embodiments are also described.

### CROSS-REFERENCE TO RELATED APPLICATIONS

The present application is a continuation-in-part of International Appl. PCT/IL 2024/050830, filed Aug. 18, 2024, which published as PCT Publication WO 2025/041129 to Gross et al., which is a continuation-in-part of U.S. application Ser. No. 18/607,638, filed Mar. 18, 2024, which published as U.S. Patent Application Publication US 2025/0058124 to Gross et al. and (a) is a continuation-in-part of U.S. application Ser. No. 18/452,229, filed Aug. 18, 2023, now U.S. Pat. No. 11,931,255, and (b) is a continuation-in-part of U.S. application Ser. No. 18/452,216, filed Aug. 18, 2023, now U.S. Pat. No. 11,975,203. All of the above-referenced applications are assigned to the assignee of the present application and incorporated herein by reference.

### FIELD OF THE APPLICATION

The present invention relates generally to surgical implants and systems, and specifically to prosthetic aortic valves and systems.

### BACKGROUND OF THE APPLICATION

Aortic heart valve replacement may be necessary to treat valve regurgitation or stenotic calcification of the leaflets. In percutaneous transluminal delivery techniques, a prosthetic aortic valve is compressed for delivery in a catheter and advanced through the descending aorta to the heart, where the prosthetic valve is deployed in the aortic valve annulus. New-onset cardiac conduction disturbances are common after transcatheter aortic valve replacement (TAVR). The most common complication is left bundle branch block (LBBB).

PCT Publication WO 2022/149130 to Gross, which is incorporated herein by reference, inter alia describes a prosthetic aortic valve, which is configured to be delivered to a native aortic valve of a patient in a constrained delivery configuration within a delivery sheath. The prosthetic aortic valve includes a frame, which includes interconnected stent struts arranged so as to define interconnected stent cells; a plurality of prosthetic leaflets coupled to the frame; a cathode and an anode, which are mechanically coupled to the frame; and a prosthetic-valve coil, which is in non-wireless electrical communication with the cathode and the anode, and is coupled to a plurality of the stent struts, running along the stent struts so as to surround a plurality of the stent cells when the prosthetic aortic valve is in an expanded fully-deployed configuration upon release from the delivery sheath.

US Patent Application Publication 2017/0258585 to Marquez et al. describes sensor-integrated prosthetic valves that can comprise a variety of features, including a plurality of valve leaflets, a frame assembly configured to support the plurality of valve leaflets and define a plurality of commissure supports terminating at an outflow end of the prosthetic valve, a sensor device associated with the frame assembly and configured to generate a sensor signal, for example, a sensor signal indicating deflection of one or more of the plurality of commissure supports, and a transmitter assembly configured to receive the sensor signal from the sensor device and wirelessly transmit a transmission signal that is based at least in part on the sensor signal.

U.S. Pat. No. 9,326,854 to Casley et al. describes medical device delivery assemblies. The assembly may include a catheter-based delivery system. The assembly may include a pacing element to pace a patient's heart before, during, or after a procedure. The pacing element may be a detachable, implanting pacing element. The pacing element may be an implantable pacemaker and the implantable pacemaker may be disposed on a catheter-based delivery system. The assembly may include a prosthetic heart valve with one or more pacing elements on it. The pacing element may include a pacing strip or strips. These strips may be conductive or insulative. These strips may prevent, treat, or correct abnormal electrical communication in a heart.

U.S. Pat. No. 11,331,476 to Capek et al. describes a method including delivering to a native valve annulus (e.g., a native mitral valve annulus) of a heart a prosthetic heart valve having a body expandable from a collapsed, delivery configuration to an expanded, deployed configuration. The method can further include, after the delivering, causing the prosthetic heart valve to move from the delivery configuration to the deployed configuration. With the prosthetic heart valve in its deployed configuration, an anchoring tether extending from the prosthetic heart valve can be secured to a wall of the heart. An electrode coupled to at least one of the prosthetic heart valve or the anchoring tether can then be used to at least one of pace the heart or sense a signal associated with the heart.

U.S. Pat. No. 7,643,879 to Shuros et al. describes systems and methods using a heart valve and an implantable medical device, such as for event detection and optimization of cardiac output. The cardiac management system includes a heart valve, having a physiological sensor. The physiological sensor is adapted to measure at least one of an intrinsic electrical cardiac parameter, a hemodynamic parameter or the like. The system further includes an implantable electronics unit, such as a cardiac rhythm management unit, coupled to the physiological sensor of the heart valve to receive physiological information. The electronics unit is adapted to use the received physiological information to control delivery of an electrical output to the subject.

US Patent Application Publication 2017/0304624 to Friedman et al. describes devices and methods that can be used for artificial cardiac pacing and/or resynchronization. For example, this patent provides improved electrodes for stimulating and sensing electrical activity of the heart, and provides pacing and resynchronization systems incorporating such electrodes. While the devices and methods provided in this patent are described primarily in the context of pacing, it should be understood that resynchronization can additionally or alternatively be performed in an analogous manner, and that the scope of this disclosure includes such subject matter.

### SUMMARY OF THE APPLICATION

Some embodiments of the present invention provide a prosthetic cardiac valve, which is configured to be implanted in a native valve of a patient, and which comprises a plurality of prosthetic leaflets, a frame, and one or more electrodes, including a cathode and an anode, mechanically coupled to the frame. The prosthetic cardiac valve further comprises a prosthetic-valve coil, which is in non-wireless electrical communication with the cathode and the anode.

For some applications, the prosthetic cardiac valve further comprises circuitry, which is configured to apply pacing to the heart and/or sense cardiac electrical activity using the one or more electrodes. For example, in configurations in which the prosthetic cardiac valve is a prosthetic aortic valve, the pacing may be applied temporarily for up to several weeks after implantation of the prosthetic aortic valve, typically using an external control unit to continuously provide power, or applied longer-term, in which case the prosthetic aortic valve may further comprise an energy storage module, e.g., comprising a battery, which may be periodically charged using the external control unit. Further alternatively or additionally, for some applications, the circuitry is configured to apply rapid pacing during an invasive structural heart procedure, such as an implantation procedure, such as a transcatheter aortic valve replacement (TAVR)-in-TAVR procedure in which the first TAVR comprises the prosthetic aortic valve.

There is therefore provided, in accordance with an application of the present invention, a prosthetic cardiac valve, which is configured to be delivered to a patient in a constrained delivery configuration, and which includes:
a frame, which defines a central longitudinal axis when the prosthetic cardiac valve is in an expanded deployment configuration, and which includes interconnected stent struts arranged so as to define interconnected stent cells that include upstream-most stent cells and non-upstream-most stent cells;
a plurality of prosthetic leaflets coupled to the frame so as to allow blood flow in a downstream direction and inhibit blood flow in an upstream direction; and
first and second electrodes, which are disposed at or near respective upstream peaks of first and second ones of the non-upstream-most stent cells, respectively.

For some applications, the first and the second electrodes are disposed at a common axial position along the central longitudinal axis when the prosthetic cardiac valve is in the expanded deployment configuration.

For some applications, the frame is shaped such that when the prosthetic cardiac valve is in the expanded deployment configuration after deployment at a native cardiac valve, the first and the second electrodes are disposed at a height of an annulus of the native cardiac valve.

For some applications, the first and the second electrodes are disposed at different respective axial positions along the central longitudinal axis when the prosthetic cardiac valve is in the expanded deployment configuration.

For some applications, the first and the second electrodes are disposed at a common angular location with respect to the central longitudinal axis when the prosthetic cardiac valve is in the expanded deployment configuration.

For some applications, the prosthetic cardiac valve further includes a third electrode, which is disposed at or near an upstream peak of a third one of the non-upstream-most stent cells.

For some applications, the first, the second, and the third electrodes are disposed at a common axial position along the central longitudinal axis when the prosthetic cardiac valve is in the expanded deployment configuration.

For some applications, the first and the second non-upstream-most stent cells are located in an upstream half of the frame.

For some applications:
the first and the second electrodes are disposed at respective axial positions along the central longitudinal axis when the prosthetic cardiac valve is in the expanded deployment configuration, the axial positions the same or differing from one another, and
each of the axial positions is at a downstream-end distance from a downstream end of the stent cells and is at an upstream-end distance from an upstream end of the stent cells,
the downstream-end distance equals at least 20% of a total axial length of the stent cells measured between the downstream end and the upstream end of the stent cells,
the upstream-end distance equals at least 20% of the total axial length of the stent cells, and
the downstream-end distance, the upstream-end distance, and the total axial length of the stent cells are measured parallel to the central longitudinal axis.

For some applications, the upstream-end distance equals at least 30% of the total axial length of the stent cells.

For some applications, the downstream-end distance equals at least 30% of the total axial length of the stent cells.

For some applications, the upstream-end distance equals at least 30% of the total axial length of the stent cells.

For some applications, the first and the second electrodes are disposed at a common axial position along the central longitudinal axis when the prosthetic cardiac valve is in the expanded deployment configuration.

For some applications, the first and the second electrodes are disposed at different respective axial positions along the central longitudinal axis when the prosthetic cardiac valve is in the expanded deployment configuration.

For some applications, the prosthetic cardiac valve further includes a third electrode, which is disposed at or near an upstream peak of a third one of the non-upstream-most stent cells.

For some applications, the first, the second, and the third electrodes are disposed at a common axial position along the central longitudinal axis when the prosthetic cardiac valve is in the expanded deployment configuration.

For some applications, the frame further includes one or more delivery-tool-coupling tabs, which extend axially beyond the stent cells.

For some applications, the prosthetic cardiac valve further includes:
circuitry; and
first and second electrical leads, which electrically couple the first and the second electrodes, respectively, to the circuitry.

For some applications, the circuitry is configured to use the first electrode as an anode and the second electrode as a cathode.

For some applications, the circuitry is configured to apply pacing to a heart of the patient using the first electrode as the anode and the second electrode as the cathode.

For some applications, the circuitry is configured to sense cardiac electrical activity of the patient using the first electrode as the anode and the second electrode as the cathode.

For some applications:
the prosthetic cardiac valve further includes an outer skirt,
a portion of the stent cells is coupled to a radially inner surface of the outer skirt so that the outer skirt covers a first axial portion of the stent cells and does not cover a second axial portion of the stent cells, and an edge of the outer skirt is disposed around a circumference of the stent cells axially at a border between the first and the second axial portions of the stent cells,
the first and the second electrodes are disposed at least partially on a radially outer surface of the outer skirt, within 8 mm of the edge of the outer skirt when the prosthetic cardiac valve is in the expanded deployment configuration.

For some applications, the first and the second electrodes are disposed at least partially on the radially outer surface of the outer skirt, within 4 mm of the edge of the outer skirt when the prosthetic cardiac valve is in the expanded deployment configuration.

For some applications, the first and the second electrodes are disposed at least partially on the radially outer surface of the outer skirt, within 2 mm of the edge of the outer skirt when the prosthetic cardiac valve is in the expanded deployment configuration.

For some applications, the first axial portion of the stent cells is downstream of the second axial portion of the stent cells.

For some applications, the outer skirt extends to or beyond an end of the stent cells.

For some applications, the prosthetic cardiac valve further includes circuitry and first and second electrical leads, which:
electrically couple the first and the second electrodes, respectively, to the circuitry, and
are coupled to interconnected stent struts of the second axial portion of the stent cells, and are not coupled to interconnected stent struts of the first axial portion of the stent cells.

For some applications, the first and the second electrical leads run along respective portions of the interconnected stent struts to which the first and the second electrical leads are respectively coupled.

For some applications:
the prosthetic cardiac valve further includes an inner skirt,
at least a portion of the stent cells is coupled to a radially outer surface of the inner skirt so that the inner skirt at least partially covers the second axial portion of the stent cells, and
the first and the second electrical leads are coupled to at least a portion of the interconnected stent struts covered by the inner skirt.

For some applications, the prosthetic cardiac valve is a prosthetic aortic valve. For some applications, the prosthetic cardiac valve is a prosthetic atrioventricular valve.

For some applications, the prosthetic atrioventricular valve is a prosthetic tricuspid valve.

For some applications, the prosthetic atrioventricular valve is a prosthetic mitral valve.

For some applications, the prosthetic cardiac valve is a prosthetic pulmonary valve.

For some applications, the prosthetic cardiac valve is a prosthetic caval valve. There is further provided, in accordance with an application of the present invention, a prosthetic cardiac valve, which is configured to be delivered to a patient in a constrained delivery configuration, and which includes:
a frame, which defines a central longitudinal axis when the prosthetic cardiac valve is in an expanded deployment configuration, and which includes interconnected stent struts arranged so as to define interconnected stent cells, which include upstream interconnected stent cells, which are located in an upstream half of the frame and include upstream-most stent cells;
a plurality of prosthetic leaflets coupled to the frame so as to allow blood flow in a downstream direction and inhibit blood flow in an upstream direction; and
first and second electrodes, which are coupled to the frame and not disposed at upstream peaks of the upstream-most stent cells.

For some applications, the first and the second electrodes are disposed within 8 mm of respective upstream peaks of first and second ones of the upstream-most stent cells, respectively.

For some applications, the first and the second electrodes are disposed at 8 mm from respective upstream peaks of first and second ones of the upstream-most stent cells, respectively.

For some applications, the first and the second electrodes are disposed at a common axial position along the central longitudinal axis when the prosthetic cardiac valve is in the expanded deployment configuration.

For some applications, the frame is shaped such that when the prosthetic cardiac valve is in the expanded deployment configuration after deployment at a native cardiac valve, the first and the second electrodes are disposed at a height of an annulus of the native cardiac valve.

For some applications, the prosthetic cardiac valve further includes a third electrode, which is coupled to the frame and not disposed at an upstream peak of the upstream-most stent cells.

For some applications, the first, the second, and the third electrodes are disposed at a common axial position along the central longitudinal axis when the prosthetic cardiac valve is in the expanded deployment configuration.

For some applications, the frame further includes one or more delivery-tool-coupling tabs, which extend axially beyond the interconnected stent cells.

For some applications, the prosthetic cardiac valve further includes:
circuitry; and
first and second electrical leads, which electrically couple the first and the second electrodes, respectively, to the circuitry.

For some applications, the circuitry is configured to use the first electrode as an anode and the second electrode as a cathode.

For some applications, the circuitry is configured to apply pacing to a heart of the patient using the first electrode as the anode and the second electrode as the cathode.

For some applications, the circuitry is configured to sense cardiac electrical activity of the patient using the first electrode as the anode and the second electrode as the cathode.

For some applications, the prosthetic cardiac valve is a prosthetic aortic valve.

For some applications, the prosthetic cardiac valve is a prosthetic atrioventricular valve.

For some applications, the prosthetic atrioventricular valve is a prosthetic tricuspid valve.

For some applications, the prosthetic atrioventricular valve is a prosthetic mitral valve.

For some applications, the prosthetic cardiac valve is a prosthetic pulmonary valve.

For some applications, the prosthetic cardiac valve is a prosthetic caval valve.

There is still further provided, in accordance with an application of the present invention, a prosthetic cardiac valve, which is configured to be delivered to a patient in a constrained delivery configuration, and which includes:
a frame, which defines a central longitudinal axis when the prosthetic cardiac valve is in an expanded deployment configuration, and which includes interconnected stent struts arranged so as to define interconnected stent cells;
a plurality of prosthetic leaflets coupled to the frame so as to allow blood flow in a downstream direction and inhibit blood flow in an upstream direction;
an outer skirt, wherein a portion of the stent cells is coupled to a radially inner surface of the outer skirt so that the outer skirt covers a first axial portion of the stent cells and does not cover a second axial portion of the stent cells, and an edge of the outer skirt is disposed around a circumference of the stent cells axially at a border between the first and the second axial portions of the stent cells; and
first and second electrodes, the first electrode is disposed at least partially on a radially outer surface of the outer skirt, within 8 mm of the edge of the outer skirt when the prosthetic cardiac valve is in the expanded deployment configuration.

For some applications, the first electrode is disposed at least partially on the radially outer surface of the outer skirt, within 4 mm of the edge of the outer skirt when the prosthetic cardiac valve is in the expanded deployment configuration.

For some applications, the first electrode is disposed at least partially on the radially outer surface of the outer skirt, within 2 mm of the edge of the outer skirt when the prosthetic cardiac valve is in the expanded deployment configuration.

For some applications, the first axial portion of the stent cells is downstream of the second axial portion of the stent cells.

For some applications, the outer skirt extends to or beyond either the upstream end or the downstream end of the interconnected stent cells.

For some applications, the frame further includes one or more delivery-tool-coupling tabs, which extend axially beyond the interconnected stent cells.

For some applications:
the prosthetic cardiac valve further includes circuitry and first and second electrical leads, which electrically couple the first and the second electrodes, respectively, to the circuitry, and
the first electrode is coupled to interconnected stent struts of the second axial portion of the stent cells, and is not coupled to interconnected stent struts of the first axial portion of the stent cells.

For some applications, the first electrical lead runs along a portion of the interconnected stent struts to which the first electrical lead is coupled.

For some applications:
the prosthetic cardiac valve further includes an inner skirt,
at least a portion of the stent cells is coupled to a radially outer surface of the inner skirt so that the inner skirt at least partially covers the second axial portion of the stent cells, and
the first electrical lead is coupled to at least a portion of the interconnected stent struts covered by the inner skirt.

For some applications, the second electrode is disposed at least partially on the radially outer surface of the outer skirt, within 8 mm of the edge of the outer skirt when the prosthetic cardiac valve is in the expanded deployment configuration.

For some applications, the first and the second electrodes are disposed at a common axial position along the central longitudinal axis when the prosthetic cardiac valve is in the expanded deployment configuration.

For some applications, the frame is shaped such that when the prosthetic cardiac valve is in the expanded deployment configuration after deployment at a native cardiac valve, the first and the second electrodes are disposed at a height of an annulus of the native cardiac valve.

For some applications, the prosthetic cardiac valve further includes a third electrode, which is disposed at least partially on the radially outer surface of the outer skirt, within 8 mm of the edge of the outer skirt disposed at an axial position along the central longitudinal axis when the prosthetic cardiac valve is in the expanded deployment configuration.

For some applications, the first, the second, and the third electrodes are disposed at a common axial position along the central longitudinal axis when the prosthetic cardiac valve is in the expanded deployment configuration.

For some applications, the first and the second electrodes are disposed at or near respective peaks of first and second ones of the stent cells, respectively.

For some applications, the first and the second electrodes are coupled to respective interconnected stent struts of first and second ones of the stent cells, respectively.

For some applications:
the first and the second electrodes are disposed at respective axial positions along the central longitudinal axis when the prosthetic cardiac valve is in the expanded deployment configuration, the axial positions the same or differing from one another, and
each of the axial positions is at a downstream-end distance from a downstream end of the stent cells and is at an upstream-end distance from an upstream end of the stent cells,
the downstream-end distance equals at least 20% of a total axial length of the stent cells measured between downstream end 748 and upstream end 746 of the stent cells,
the upstream-end distance equals at least 20% of the total axial length of the stent cells, and
the downstream-end distance, the upstream-end distance, and the total axial length of the stent cells are measured parallel to the central longitudinal axis.

For some applications, the upstream-end distance equals at least 30% of the total axial length of the stent cells.

For some applications, the downstream-end distance equals at least 30% of the total axial length of the stent cells.

For some applications, the upstream-end distance equals at least 30% of the total axial length of the stent cells.

For some applications, the first and the second electrodes are disposed at a common axial position along the central longitudinal axis when the prosthetic cardiac valve is in the expanded deployment configuration.

For some applications, the first and the second electrodes are disposed at different respective axial positions along the central longitudinal axis when the prosthetic cardiac valve is in the expanded deployment configuration.

For some applications, the prosthetic cardiac valve further includes:
circuitry; and
first and second electrical leads, which electrically couple the first and the second electrodes, respectively, to the circuitry.

For some applications, the circuitry is configured to use one of the first and the second electrodes as an anode and the other of the first and the second electrodes as a cathode.

For some applications, the circuitry is configured to apply pacing to a heart of the patient the one of the first and the second electrodes as the anode and the other of the first and the second electrodes as the cathode.

For some applications, the circuitry is configured to sense cardiac electrical activity of the patient using the one of the first and the second electrodes as the anode and the other of the first and the second electrodes as the cathode.

For some applications, the prosthetic cardiac valve is a prosthetic aortic valve. For some applications, the prosthetic cardiac valve is a prosthetic atrioventricular valve.

For some applications, the prosthetic atrioventricular valve is a prosthetic tricuspid valve.

For some applications, the prosthetic atrioventricular valve is a prosthetic mitral valve.

For some applications, the prosthetic cardiac valve is a prosthetic pulmonary valve.

For some applications, the prosthetic cardiac valve is a prosthetic caval valve.

There is additionally provided, in accordance with an application of the present invention, a prosthetic atrioventricular valve, which is configured to be delivered to a native atrioventricular valve of a heart of a patient in a constrained delivery configuration, and which includes:
a frame;
a plurality of prosthetic leaflets coupled to the frame so as to allow blood flow in a downstream direction and inhibit blood flow in an upstream direction;
a plurality of anchoring arms; and
first and second electrodes, which are coupled to first and second ones of the anchoring arms, respectively,
wherein, when the prosthetic atrioventricular valve is in an expanded deployment configuration, the anchoring arms extend in a radially outward and upstream direction, so as to engage one or more portions of the heart and thereby help hold the prosthetic atrioventricular valve in place upon deployment, the one or more portions selected from the group consisting of: chordae tendineae of the heart, and downstream surfaces of leaflets of the native atrioventricular valve.

For some applications, the first and the second electrodes are coupled to the first and the second anchoring arms, respectively, at or within 5 mm of respective free ends of the first and the second anchoring arms, respectively.

For some applications, the first and the second electrodes are coupled to the first and the second anchoring arms, respectively, at or within 3 mm of the respective free ends of the first and the second anchoring arms, respectively.

For some applications, the prosthetic atrioventricular valve further includes a third electrode, which is coupled to a third one of the anchoring arms.

For some applications, the prosthetic atrioventricular valve further includes:
circuitry; and
first and second electrical leads, which electrically couple the first and the second electrodes, respectively, to the circuitry.

For some applications, the circuitry is configured to use the first electrode as an anode and the second electrode as a cathode.

For some applications, the circuitry is configured to apply pacing to the heart using the first electrode as the anode and the second electrode as the cathode.

For some applications, the circuitry is configured to sense cardiac electrical activity of the patient using the first electrode as the anode and the second electrode as the cathode.

For some applications, the anchoring arms are configured, when the prosthetic atrioventricular valve is in the expanded deployment configuration, to bring the first and the second electrodes into contact with one or more surfaces selected from the group consisting of: one or more downstream surfaces of one or more respective leaflets of the native atrioventricular valve, and a ventricular wall in a subvalvular space of the native atrioventricular valve.

For some applications, the prosthetic atrioventricular valve is a prosthetic tricuspid valve.

For some applications, the prosthetic atrioventricular valve is a prosthetic mitral valve.

There is yet additionally provided, in accordance with an application of the present invention, a method including:
delivering a prosthetic atrioventricular valve to a native atrioventricular valve of a heart of a patient in a constrained delivery configuration, the prosthetic atrioventricular valve including a frame, a plurality of prosthetic leaflets coupled to the frame, a plurality of anchoring arms, and first and second electrodes, which are coupled to first and second ones of the anchoring arms, respectively; and
transitioning the prosthetic atrioventricular valve to an expanded deployment configuration in which (a) the plurality of prosthetic leaflets allow blood flow in a downstream direction and inhibit blood flow in an upstream direction, and (b) the anchoring arms extend in a radially outward and upstream direction, so as to engage one or more portions of the heart and thereby help hold the prosthetic atrioventricular valve in place upon deployment, the one or more portions selected from the group consisting of: chordae tendineae of the heart, and downstream surfaces of leaflets of the native atrioventricular valve.

For some applications, the first and the second electrodes are coupled to the first and the second anchoring arms, respectively, at or within 5 mm of respective free ends of the first and the second anchoring arms, respectively.

For some applications, the first and the second electrodes are coupled to the first and the second anchoring arms, respectively, at or within 3 mm of the respective free ends of the first and the second anchoring arms, respectively.

For some applications, the prosthetic atrioventricular valve further includes a third electrode, which is coupled to a third one of the anchoring arms.

For some applications, the prosthetic atrioventricular valve further includes circuitry; and first and second electrical leads, which electrically couple the first and the second electrodes, respectively, to the circuitry.

For some applications, the method further includes activating the circuitry to use one of the first and the second electrodes as an anode and the other of the first and the second electrodes as a cathode.

For some applications, activating the circuitry includes activating the circuitry to apply pacing to the heart using the first electrode as the anode and the second electrode as the cathode.

For some applications, activating the circuitry includes activating the circuitry to sense cardiac electrical activity of the patient using the first electrode as the anode and the second electrode as the cathode.

For some applications, transitioning includes transitioning the prosthetic atrioventricular valve to the expanded deployment configuration in which the anchoring arms bring the first and the second electrodes into contact with one or more surfaces selected from the group consisting of: one or more downstream surfaces of one or more respective leaflets of the native atrioventricular valve, and a ventricular wall in a subvalvular space of the native atrioventricular valve.

For some applications, the prosthetic atrioventricular valve is a prosthetic tricuspid valve.

For some applications, the prosthetic atrioventricular valve is a prosthetic mitral valve.

There is also provided, in accordance with an application of the present invention, a prosthetic atrioventricular valve, which is configured to be delivered to a native atrioventricular valve of a heart of a patient in a constrained delivery configuration, and which includes:
a frame;
a plurality of prosthetic leaflets coupled to the frame so as to allow blood flow in a downstream direction and inhibit blood flow in an upstream direction;
first and second electrodes,
wherein the prosthetic atrioventricular valve is configured, when in an expanded deployment configuration, to bring the first electrode into contact with one or more surfaces selected from the group consisting of: one or more downstream surfaces of one or more respective leaflets of the native atrioventricular valve, and a ventricular wall in a subvalvular space of the native atrioventricular valve.

For some applications, the one or more surfaces are the one or more downstream surfaces of the one or more respective leaflets of the native atrioventricular valve.

For some applications, the one or more surfaces are the ventricular wall in the subvalvular space of the native atrioventricular valve.

For some applications:
the prosthetic atrioventricular valve further includes a plurality of anchoring arms,
the first electrode is coupled to one of the anchoring arms, and
when the prosthetic atrioventricular valve is in the expanded deployment configuration, the anchoring arms extend in a radially outward and upstream direction, so as to engage one or more portions of the heart and thereby help hold the prosthetic atrioventricular valve in place upon deployment, the one or more portions selected from the group consisting of: chordae tendineae of the heart, and downstream surfaces of leaflets of the native atrioventricular valve.

For some applications, the first electrode is coupled to the one of the anchoring arms at or within 5 mm of a free end of the one of the anchoring arms.

For some applications, the first electrode is coupled to the one of the anchoring arms at or within 3 mm of the free end of the one of the anchoring arms.

For some applications, the prosthetic atrioventricular valve is configured, when in the expanded deployment configuration, to bring the first and the second electrodes into contact with the one or more surfaces.

For some applications, the prosthetic atrioventricular valve further includes a third electrode, the prosthetic atrioventricular valve is configured, when in the expanded deployment configuration, to bring the first, the second, and the third electrodes into contact with the one or more surfaces.

For some applications, the prosthetic atrioventricular valve further includes:
circuitry; and
first and second electrical leads, which electrically couple the first and the second electrodes, respectively, to the circuitry.

For some applications, the circuitry is configured to use one of the first and the second electrodes as an anode and the other of the first and the second electrodes as a cathode.

For some applications, the circuitry is configured to apply pacing to the heart using the one of the first and the second electrodes as the anode and the other of the first and the second electrodes as the cathode.

For some applications, the circuitry is configured to sense cardiac electrical activity of the patient using the one of the first and the second electrodes as the anode and the other of the first and the second electrodes as the cathode.

For some applications, the prosthetic atrioventricular valve is a prosthetic tricuspid valve.

For some applications, the prosthetic atrioventricular valve is a prosthetic mitral valve.

There is further provided, in accordance with an application of the present invention, a method including:
delivering a prosthetic atrioventricular valve to a native atrioventricular valve of a heart of a patient in a constrained delivery configuration, the prosthetic atrioventricular valve including a frame, a plurality of prosthetic leaflets coupled to the frame, and first and second electrodes; and
transitioning the prosthetic atrioventricular valve to an expanded deployment configuration in which (a) the plurality of prosthetic leaflets allow blood flow in a downstream direction and inhibit blood flow in an upstream direction, and (b) the first electrode is in contact with one or more surfaces selected from the group consisting of: one or more downstream surfaces of one or more respective leaflets of the native atrioventricular valve, and a ventricular wall in a subvalvular space of the native atrioventricular valve.

For some applications, the one or more surfaces are the one or more downstream surfaces of the one or more respective leaflets of the native atrioventricular valve.

For some applications, the one or more surfaces are the ventricular wall in the subvalvular space of the native atrioventricular valve.

For some applications:
the prosthetic atrioventricular valve further includes a plurality of anchoring arms,
the first electrode is coupled to one of the anchoring arms, and
transitioning includes transitioning the prosthetic atrioventricular valve to the expanded deployment configuration in which the anchoring arms extend in a radially outward and upstream direction, so as to engage one or more portions of the heart and thereby help hold the prosthetic atrioventricular valve in place upon deployment, the one or more portions selected from the group consisting of: chordae tendineae of the heart, and downstream surfaces of leaflets of the native atrioventricular valve.

For some applications, the first electrode is coupled to the one of the anchoring arms at or within 5 mm of a free end of the one of the anchoring arms.

For some applications, the first electrode is coupled to the one of the anchoring arms at or within 3 mm of the free end of the one of the anchoring arms.

For some applications, transitioning includes transitioning the prosthetic atrioventricular valve to the expanded deployment configuration in which the first and the second electrodes are in contact with the one or more surfaces.

For some applications, the prosthetic atrioventricular valve further includes a third electrode, and transitioning includes transitioning the prosthetic atrioventricular valve to the expanded deployment configuration in which the first, the second, and the third electrodes are in contact with the one or more surfaces.

For some applications, the method further includes activating circuitry, which is electrically coupled to the first and the second electrodes, to use one of the first and the second electrodes as an anode and the other of the first and the second electrodes as a cathode.

For some applications, activating the circuitry includes activating the circuitry to apply pacing to the heart using the one of the first and the second electrodes as the anode and the other of the first and the second electrodes as the cathode.

For some applications, activating the circuitry includes activating the circuitry to sense cardiac electrical activity of the patient using the one of the first and the second electrodes as the anode and the other of the first and the second electrodes as the cathode.

For some applications, the native atrioventricular valve is a native tricuspid valve.

For some applications, the native atrioventricular valve is a native mitral valve. There is still further provided, in accordance with an application of the present invention, a prosthetic cardiac valve, which is configured to be delivered to a patient in a constrained delivery configuration, and which includes:
an inner frame, which includes inner interconnected stent struts arranged so as to define inner interconnected stent cells;
an outer frame, which includes outer interconnected stent struts arranged so as to define outer interconnected stent cells, and which is coupled to the inner frame surrounding at least an axial portion of the inner frame, wherein the prosthetic cardiac valve is configured such that when the prosthetic cardiac valve is in an expanded deployment configuration, (a) the inner frame applies an outwardly-directed radial force to assist with fixation of the outer frame to native anatomy of the patient, and (b) the inner and the outer frames together define a central longitudinal axis;
a plurality of prosthetic leaflets coupled to the inner frame so as to allow blood flow in a downstream direction and inhibit blood flow in an upstream direction; and
first and second electrodes, wherein the first electrode is coupled to the outer frame.

For some applications, the second electrode is coupled to the outer frame.

For some applications, the first and the second electrodes are disposed at a common axial position along the central longitudinal axis when the prosthetic cardiac valve is in the expanded deployment configuration.

For some applications, the inner and the outer frames is shaped such that when the prosthetic cardiac valve is in the expanded deployment configuration after deployment at a native cardiac valve, the first and the second electrodes are disposed at a height of an annulus of the native cardiac valve.

For some applications, the prosthetic cardiac valve further includes a third electrode, which is coupled to the outer frame.

For some applications, the first, the second, and the third electrodes are disposed at a common axial position along the central longitudinal axis when the prosthetic cardiac valve is in the expanded deployment configuration.

For some applications, the prosthetic cardiac valve further includes:
circuitry; and
first and second electrical leads, which electrically couple the first and the second electrodes, respectively, to the circuitry.

For some applications, the circuitry is configured to use one of the first and the second electrodes as an anode and the other of the first and the second electrodes as a cathode.

For some applications, the circuitry is configured to apply pacing to a heart of the patient the one of the first and the second electrodes as the anode and the other of the first and the second electrodes as the cathode.

For some applications, the circuitry is configured to sense cardiac electrical activity of the patient using the one of the first and the second electrodes as the anode and the other of the first and the second electrodes as the cathode.

For some applications, the prosthetic cardiac valve is a prosthetic aortic valve. For some applications, the prosthetic cardiac valve is a prosthetic atrioventricular valve.

For some applications, the prosthetic atrioventricular valve is a prosthetic tricuspid valve.

For some applications, the prosthetic atrioventricular valve is a prosthetic mitral valve.

For some applications, the prosthetic cardiac valve is a prosthetic pulmonary valve.

For some applications, the prosthetic cardiac valve is a prosthetic caval valve.

There is additionally provided, in accordance with an application of the present invention, a prosthetic cardiac valve, which is configured to be delivered to a native cardiac valve of a heart of a patient in a constrained delivery configuration, and which includes:
a frame, which defines a central longitudinal axis when the prosthetic cardiac valve is in an expanded deployment configuration;
a plurality of prosthetic leaflets coupled to the frame so as to allow blood flow in a downstream direction and inhibit blood flow in an upstream direction when the prosthetic cardiac valve is in the expanded deployment configuration;
a plurality of electrodes mechanically coupled to the frame at a common axial position along the central longitudinal axis when the prosthetic cardiac valve is in the expanded deployment configuration; and
circuitry, which is electrically coupled to the electrodes, and which is configured to use one or more of the electrodes as one or more anodes and one or more of the other electrodes as one or more cathodes.

For some applications, the prosthetic cardiac valve includes only the plurality of electrodes mechanically coupled to the frame at the common axial position along the central longitudinal axis when the prosthetic cardiac valve is in the expanded deployment configuration.

For some applications, the circuitry is configured to use only the one or more of the electrodes as the one or more anodes and only the one or more of the other electrodes as the one or more cathodes.

For some applications, the circuitry is configured to apply pacing to the heart using the one or more of the electrodes as the one or more anodes and the one or more of the other electrodes as the one or more cathodes.

For some applications, the circuitry is configured to sense cardiac electrical activity of the patient using the one or more of the electrodes as the one or more anodes and the one or more of the other electrodes as the one or more cathodes.

The present invention will be more fully understood from the following detailed description of embodiments thereof, taken together with the drawings, in which:

### BRIEF DESCRIPTION OF THE DRAWINGS

FIGS. 1A and 1B are schematic illustrations a prosthetic aortic valve, in accordance with an application of the present invention;

FIG. 2 is a schematic illustration of a valve prosthesis system and the prosthetic aortic valve of FIGS. 1A-B implanted in a body of a patient, in accordance with an application of the present invention;

FIGS. 3A-C are schematic illustrations of a printed circuit board (PCB), an electrical lead, and electrodes, in accordance with an application of the present invention;

FIGS. 3D and 3E are schematic illustrations of a portion of the prosthetic aortic valve of FIGS. 1A-B and the PCB of FIGS. 3A-C coupled to stent struts of the prosthetic aortic valve, in accordance with respective applications of the present invention;

FIGS. 3F and 3G are schematic illustrations additional configurations of the PCB of FIGS. 3A-C, in accordance with respective applications of the present invention;

FIG. 3H is a schematic illustration of another configuration of a frame of the prosthetic aortic valve of FIGS. 1A-B and the PCB of FIGS. 3A-C coupled to the frame, in accordance with an application of the present invention;

FIG. 3I is a schematic illustration of yet another configuration of the PCB of FIGS. 3A-C, in accordance with an application of the present invention;

FIG. 4 is a schematic illustration of a portion of the prosthetic aortic valve of FIGS. 1A-B in a constrained delivery configuration within a delivery sheath, in accordance with an application of the present invention;

FIG. 5 is a schematic illustration of a prosthetic atrioventricular valve, in accordance with an application of the present invention;

FIG. 6 is a schematic illustration of an external control unit of a valve prosthesis system comprising the prosthetic aortic valve of FIGS. 1A-B, in accordance with an application of the present invention;

FIGS. 7A-B are schematic illustrations of two configurations of a prosthetic atrioventricular valve, in accordance with respective applications of the present invention;

FIG. 8 is a schematic illustration of the prosthetic atrioventricular valve of FIGS. 7A-B implanted in a native atrioventricular valve, in accordance with an application of the present invention;

FIGS. 9A and 9B are schematic illustrations of two configurations of another prosthetic atrioventricular valve, in accordance with respective applications of the present invention; and

FIG. 10 is a schematic illustration of yet another prosthetic atrioventricular valve, in accordance with an application of the present invention.

### DETAILED DESCRIPTION OF APPLICATIONS

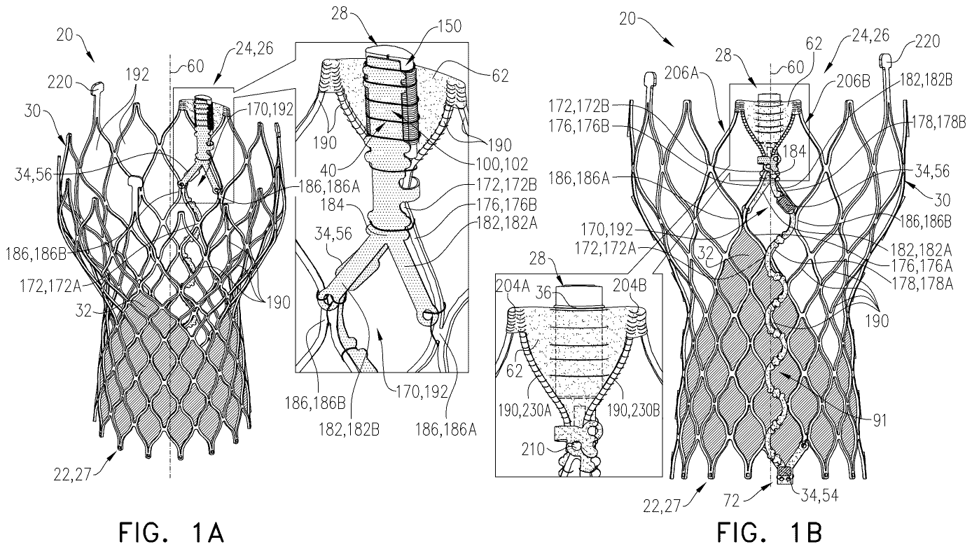

Reference is made to FIGS. 1A and 1B, which are schematic illustrations of a prosthetic aortic valve 20, in accordance with an application of the present invention. For clarity of illustration, only the closer half of prosthetic aortic valve 20 is shown in FIG. 1B.

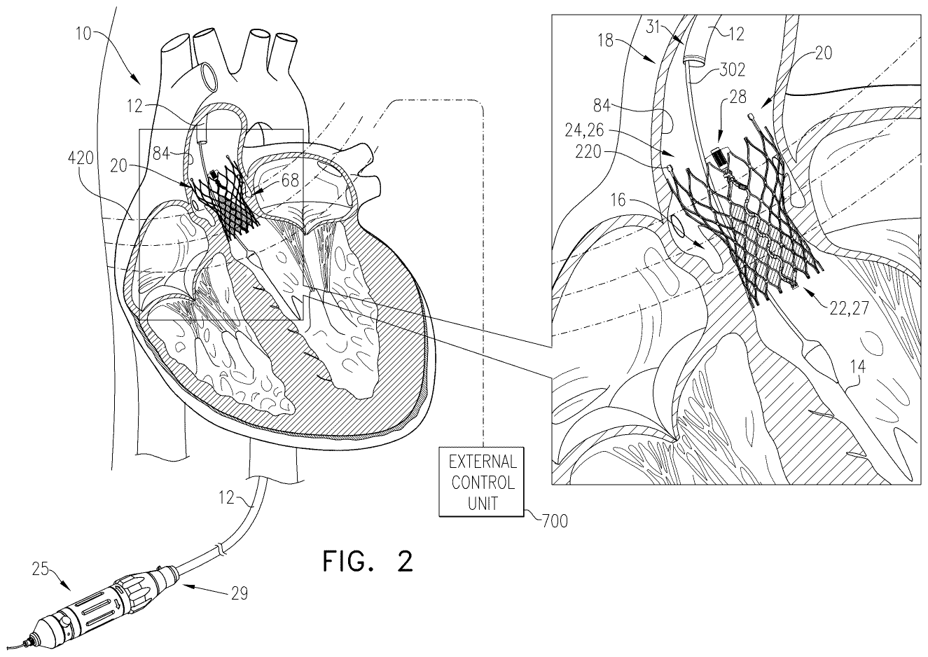

Reference is also made to FIG. 2, which is a schematic illustration of a valve prosthesis system 10 and prosthetic aortic valve 20 implanted in a body of a patient, in accordance with an application of the present invention. Valve prosthesis system 10 further comprises a delivery system 18, which typically comprises a delivery sheath 12 and is used with a guidewire 14. Delivery system 18 typically further comprises a user-control handle 25, which is disposed at (and optionally coupled to) a proximal end portion 29 of delivery sheath 12. The opposite, free end portion of delivery sheath 12 is thus a distal end portion 31 of delivery sheath 12. Prosthetic aortic valve 20 is typically configured to be delivered to a native aortic valve 16 of the patient in a constrained delivery configuration within delivery sheath 12. Distal end portion 31 of delivery sheath 12 may be a conventional tube, for example as shown. Alternatively, distal end portion 31 of delivery sheath 12 may further comprise a capsule that is moveable distally with respect to the remainder of delivery sheath 12 during deployment. All or a portion of prosthetic aortic valve 20 may be contained within the capsule. As used in the present application, including in the claims, in configurations in which distal end portion 31 comprises a capsule (or other type of holder), the distal end portion 31 of delivery sheath 12 refers to the combination of the conventional tubular portion of the sheath and the capsule. By way of example and not limitation, such a capsule is described in U.S. Pat. No. 10,888,421 to Hariton et al., which is incorporated herein by reference.

Typically, prosthetic aortic valve 20 is deployed using imaging, such as fluoroscopy, and is rotated if necessary during the deployment such that a cathode 54 is disposed against tissue of the annulus that is near the bundle of His.

Prosthetic aortic valve 20 is shown in FIGS. 1A-B and FIG. 2 in an expanded configuration. Frame 30 defines a central longitudinal axis 60 when prosthetic aortic valve 20 is in this expanded deployment configuration. Prosthetic aortic valve 20 has an upstream end 22 and a downstream end 24. Downstream end 24 may also be a proximal end 26 and upstream end 22 may also be a distal end 27, for example because proximal end 26 may be disposed in distal end portion 31 of delivery sheath 12 more proximally than distal end 27; in other words, proximal end 26 is closer to proximal end portion 29 of delivery sheath 12 than is distal end 27. For some applications, such as shown, proximal end 26 is configured to be coupled to delivery system 18 (e.g., shaped so as to define delivery-tool-coupling tabs 220, which are configured to removably couple frame 30, and thus prosthetic aortic valve 20, to delivery system 18, e.g., to a delivery shaft of delivery system 18, such as described hereinbelow). Typically, as shown in the figures, in configurations in which frame 30 is shaped to define interconnected stent cells 192, delivery-tool-coupling tabs 220 extend axially beyond the interconnected stent cells. For other applications (configuration not shown), distal end 27 is configured to be coupled to delivery system 18, such as to a capsule of the distal end portion 31 of delivery sheath 12, such as described hereinabove.

Prosthetic aortic valve 20 comprises:
a frame 30;
a plurality of prosthetic leaflets 32 coupled to frame 30 so as to allow blood flow in a downstream direction and inhibit blood flow in an upstream direction when prosthetic aortic valve 20 is in the expanded deployment configuration, such as shown in FIGS. 1A-B and 2;
an antenna 28, which is mechanically coupled to frame 30, and which comprises one or more prosthetic-valve coils 36; one or more electrodes 34, such as cathode 54 and an anode 56, coupled to frame 30; and
optionally, circuitry 40, which is electrically coupled to cathode 54, anode 56, and the one or more prosthetic-valve coils 36.

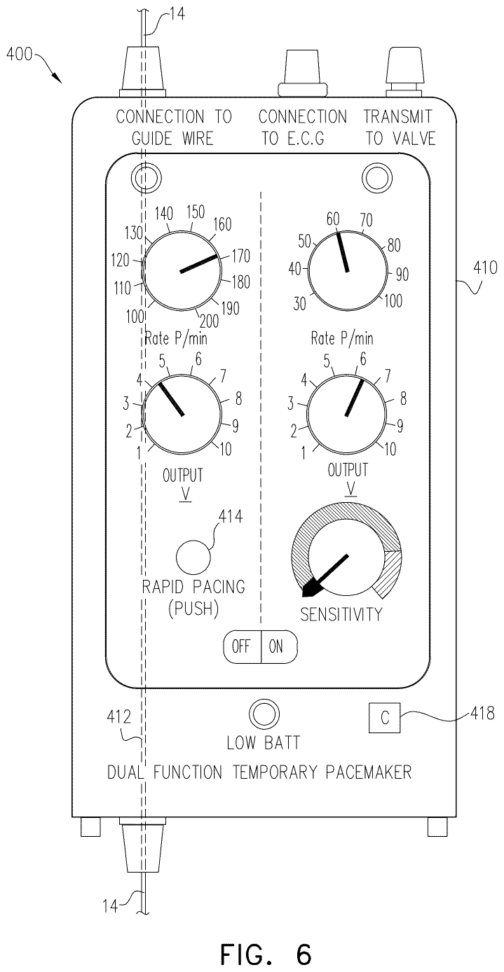

Typically, circuitry 40 is configured to apply pacing to the heart using the one or more electrodes 34. For example, the pacing may be applied temporarily for up to several weeks after implantation of prosthetic aortic valve 20 (e.g., up to one month after implantation), typically using an external control unit to continuously provide power, such as external control unit 400, described hereinbelow with reference to FIG. 6.

Alternatively, for some applications, the pacing is applied longer-term, in which case valve prosthesis system 10 may comprise an energy storage module, e.g., comprising a battery. For example, prosthetic aortic valve 20 may further comprise the energy storage module, e.g., comprising a battery, which may be periodically charged using the external control unit, which may obviate the need for the patient to constantly wear an external energy transmitter. Alternatively or additionally, for example, valve prosthesis system 10 may comprise an implantable energy storage module, e.g., comprising a battery (e.g., a rechargeable battery); for example, the energy storage module may be implantable subcutaneously. The implantable energy storage unit may provide power to prosthetic aortic valve 20 either wirelessly and/or wiredly. For example, the pacing may comprise ongoing sensing of a native electrical signal of the heart and deliverance of electrical stimulus in cases in which the native signal is unsatisfactory for timely ventricular contraction (“VVI pacing”).

Further alternatively or additionally, for some applications, circuitry 40 is configured to apply rapid pacing during an invasive structural heart procedure, such as an implantation procedure, such as a TAVR-in-TAVR procedure in which the first TAVR comprises prosthetic aortic valve 20.

For some applications, prosthetic aortic valve 20 is configured to sense cardiac electrical activity, such as reflected in an electrocardiogramand/or an intracardiac electrogram (EGM) of the patient's heart. Circuitry 40 may be configured to sense the ECG and/or EGM, or separate circuitry may be provided for sensing the ECG and/or EGM. The ECG and/or EGM sensing may be performed using all or a subset of electrodes 34 and/or one or more separate electrodes may be provided for performing the ECG and/or EGM sensing.

Each of the one or more prosthetic-valve coils 36 comprises an electrically conductive wire coated with electrical insulation.

Frame 30 typically comprises a stent or other structure, which is typically self-expanding, and may be formed by laser cutting or etching a metal alloy tube comprising, for example, stainless steel or a shape memory material such as Nitinol. For some applications, frame 30 comprises interconnected stent struts 190 arranged so as to define interconnected stent cells 192. Optionally, interconnected stent cells 192 are generally diamond-shaped, such as shown in the drawings.

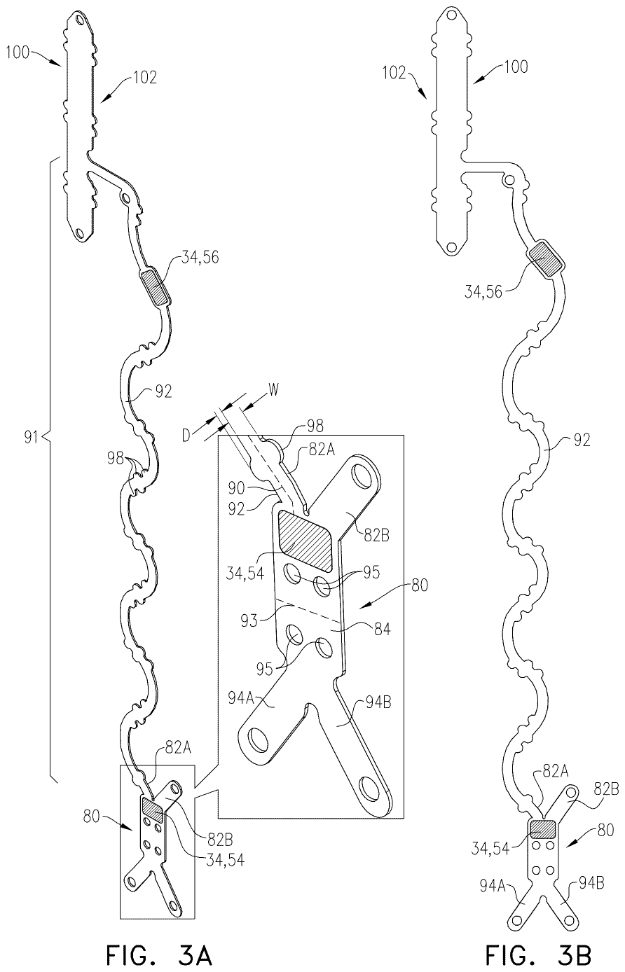

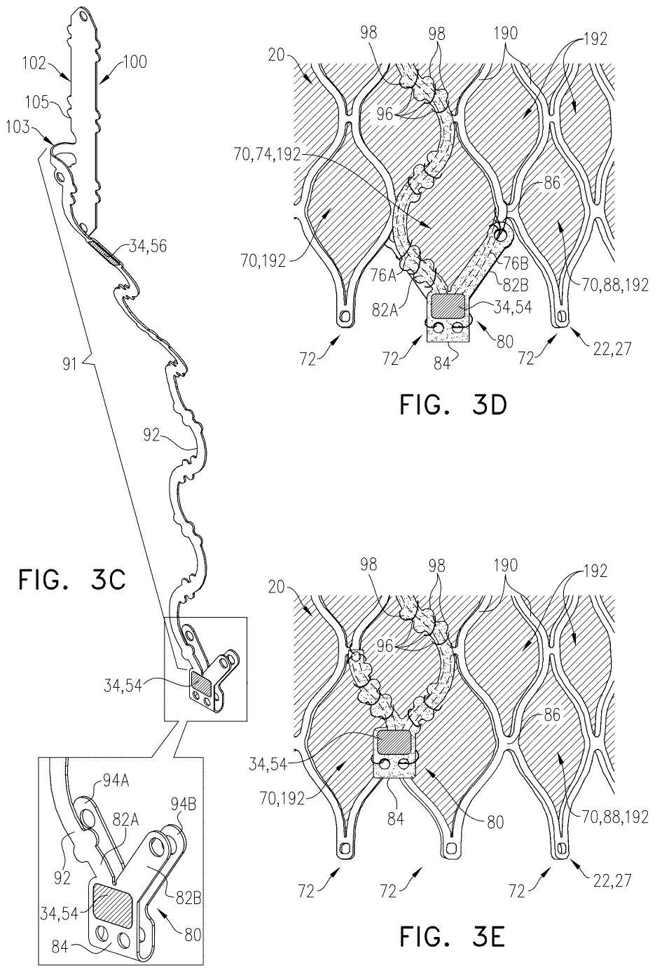

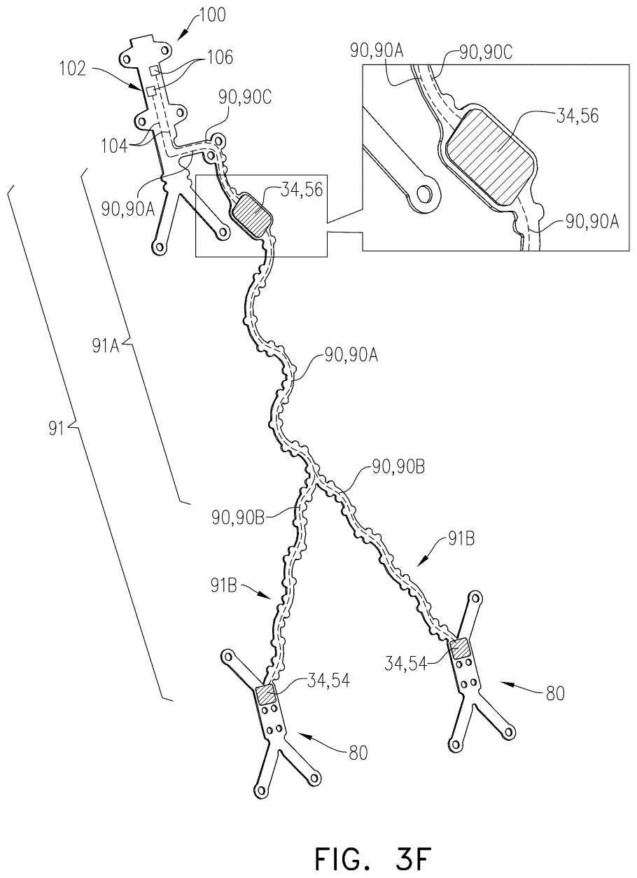

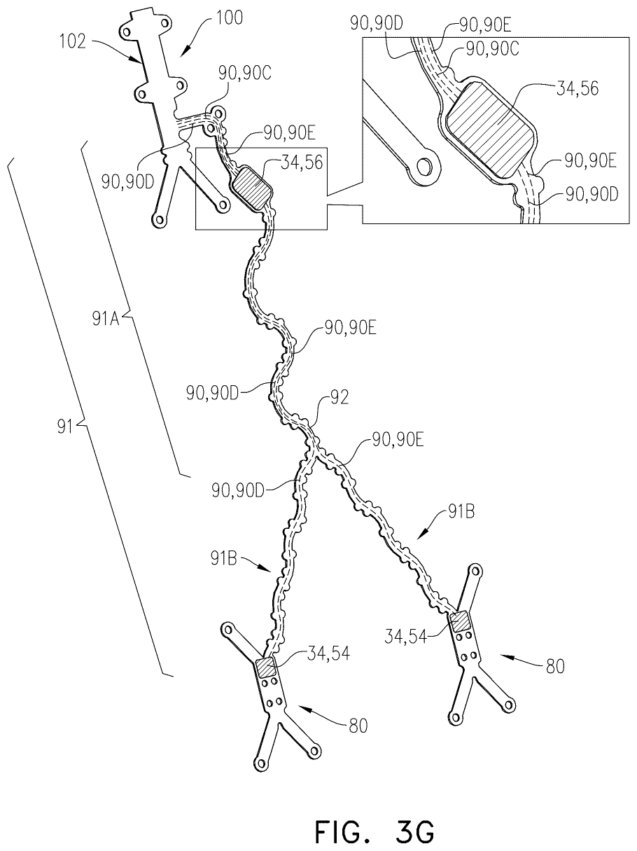

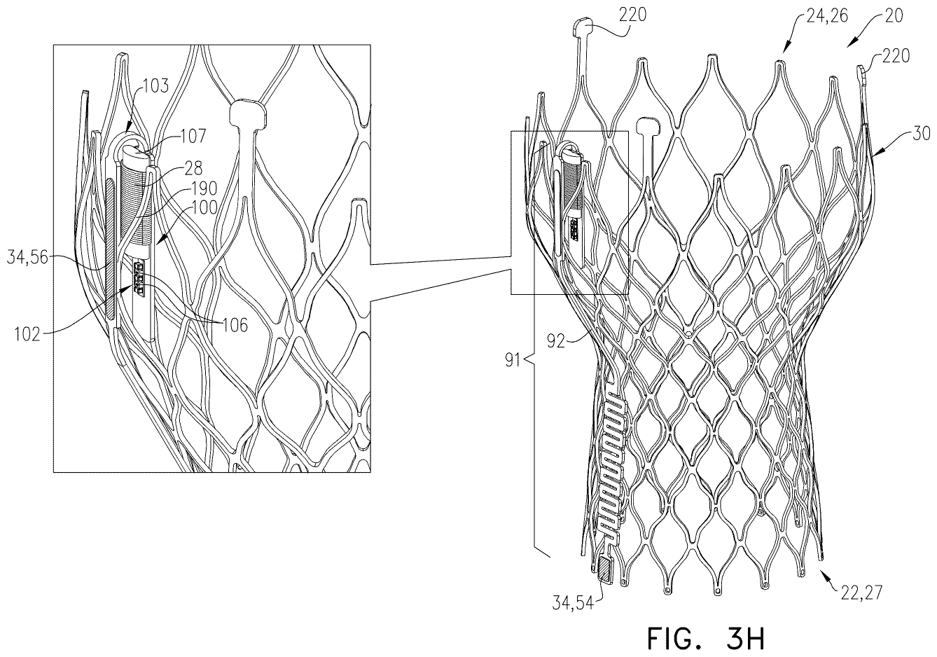

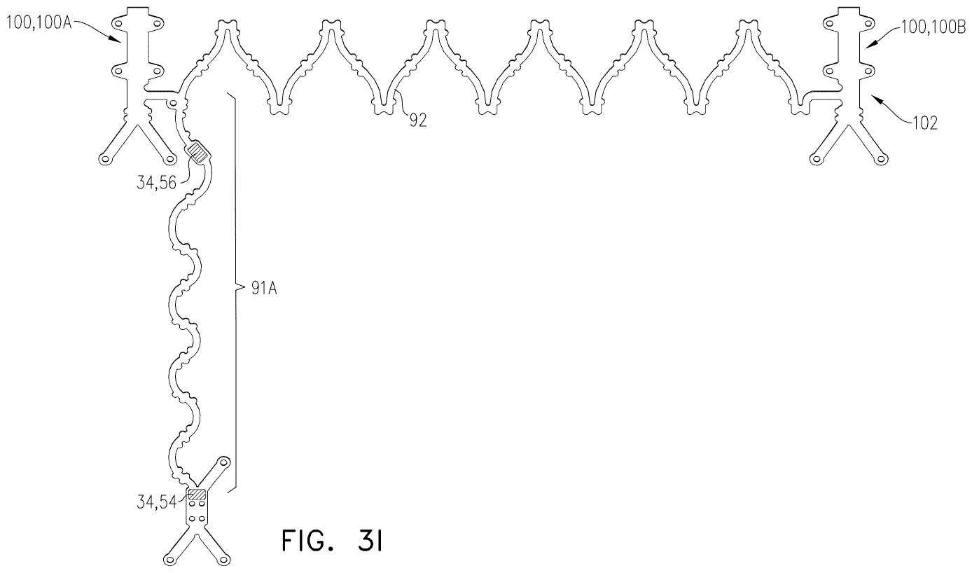

Electrodes 34 may be coupled to frame 30 in various ways. For example, the electrodes may be coupled to stent struts 190 (e.g., by stitching, by soldering, or by using the techniques described hereinbelow with reference to FIGS. 3A-G). Alternatively, the electrodes may be coupled to a skirt or other sheet of thin material that is coupled to stent struts 190, such that the electrodes are coupled to frame 30 via the skirt or other sheet. Further alternatively, the electrodes may be coupled to stent struts 190 such as shown in FIG. 3H, described hereinbelow. Still further alternatively, the electrodes may comprise a coating of conductive material that is coated on a skirt or other sheet of thin material that is coupled to stent struts 190, such that the electrodes are coupled to frame 30 via the skirt or other sheet. Other techniques for coupling the electrodes to frame 30 will be apparent to those skilled in the art who have read the present disclosure, and are within the scope of embodiments of the invention.

Typically, adjoining pairs of prosthetic leaflets 32 are attached to one another at their lateral ends to form commissures, with free edges of the prosthetic leaflets forming coaptation edges that meet one another. Prosthetic leaflets 32 typically comprise a sheet of animal pericardial tissue, such as porcine pericardial tissue, or synthetic or polymeric material. Optionally, prosthetic aortic valve 20 further comprises a skirt. Optionally, leaflets 32 are coupled to frame 30 via being coupled to the skirt, which is coupled to frame 30, or the leaflets are otherwise directly or indirectly coupled to the frame.

For some applications, cathode 54 has a thickness of at least 10 microns, no more than 200 microns, and/or between 10 and 200 microns, e.g., about 50 microns, and/or a surface area of at least 0.5 mm\hat{ }2, e.g., at least 1 mm\hat{ }2; no more than 20 mm\hat{ }2; and/or 0.5-20 mm\hat{ }2, such as 1-20 mm\hat{ }2, in order to provide adequate stimulation. For some applications, cathode 54 is coated with titanium nitride (TiN).

Typically, antenna 28 is mechanically coupled to frame 30 proximal (e.g., downstream) of prosthetic leaflets 32, such as shown. Alternatively, antenna 28 is mechanically coupled to frame 30 distal of prosthetic leaflets 32, or at least partially axially overlapping with prosthetic leaflets 32 (configurations not shown).

Reference is made to FIGS. 1A-B. For some applications:
circumferentially adjacent first and second proximal (e.g., downstream)-most stent cells 206A and 206B of interconnected stent cells 192 are joined at a cell junction 210 (labeled in FIG. 1B),
first proximal (e.g., downstream)-most stent cell 206A comprises a right proximal (e.g., downstream) strut 230A of interconnected stent struts 190, right proximal (e.g., downstream) strut 230A extending between cell junction 210 and a first proximal (e.g., downstream) peak 204A defined by first proximal (e.g., downstream)-most stent cell 206A, and
second proximal (e.g., downstream)-most stent cell 206B comprises a left proximal (e.g., downstream) strut 230B of interconnected stent struts 190, left proximal (e.g., downstream) strut 230B extending between cell junction 210 and a second proximal (e.g., downstream) peak 204B defined by second proximal (e.g., downstream)-most stent cell 206B.

For some applications, prosthetic aortic valve 20 further comprises a flexible sheet 62, which is mechanically coupled to right and left proximal (e.g., downstream) struts 230A and 230B. Optionally, flexible sheet 62 is mechanically coupled to right and left proximal (e.g., downstream) struts 230A and 230B by stitching, such as shown; alternatively or additionally, flexible sheet 62 is mechanically coupled to right and left proximal (e.g., downstream) struts 230A and 230B using alternative coupling techniques that are known in the art.

Flexible sheet 62 may comprise, for example, a polymer (e.g., polyethylene terephthalate (PET) or expanded Polytetrafluoroethylene (ePTFE)) or biological tissue, e.g., a pericardium sheet. Optionally, the material of flexible sheet 62 is woven. Optionally, the material of flexible sheet 62 comprises cloth. Flexible sheet 62 is collapsible with prosthetic aortic valve 20 when loaded into delivery sheath 12.

Antenna 28 is mechanically coupled to frame 30 at least in part by being mechanically coupled to flexible sheet 62 between right and left proximal (e.g., downstream) struts 230A and 230B. Optionally, antenna 28 is mechanically coupled flexible sheet 62 by stitching, such as shown; alternatively or additionally, antenna 28 is mechanically coupled to flexible sheet 62 using alternative coupling techniques that are known in the art. (Because flexible sheet 62 and antenna 28 are shown from outside prosthetic aortic valve 20 in FIG. 1B, flexible sheet 62 partially obscures the view of antenna 28.) Optionally, antenna 28 is mechanically coupled to frame 30 at least in part by being mechanically coupled to cell junction 210.

For some applications, flexible sheet 62 has an area of 25-100 mm\hat{ }2.

For some applications, flexible sheet 62 is coupled only to one or more interconnected stent struts 190 of each of first and second proximal (e.g., downstream)-most stent cells 206A and 206B, and not to any interconnected stent struts 190 of other stent cells of frame 30.

For some applications, flexible sheet 62 has three sides.

Typically, flexible sheet 62 is separate and distinct from material of prosthetic leaflets 32.

Reference is now made to FIGS. 3A-C, which are schematic illustrations of a printed circuit board (PCB) 92, an electrical lead 90, and electrodes 34, in accordance with an application of the present invention. PCB 92 typically comprises a polymer, such as polyimide, as is known the PCB art. PCB 92 is typically flexible.

Reference is also made to FIGS. 3D and 3E, which are schematic illustrations of a portion of prosthetic aortic valve 20 and PCB 92 coupled to stent struts 190, in accordance with respective applications of the present invention.

Reference is further made to FIGS. 3F and 3G, which are schematic illustrations additional configurations of PCB 92, in accordance with respective applications of the present invention.

Reference is still further made to FIG. 3H, which is a schematic illustration of frame 30 of prosthetic aortic valve 20 and another configuration of PCB 92 coupled to frame 30, in accordance with an application of the present invention.

Reference is additionally made to FIG. 3I, which is a schematic illustration of yet another configuration of PCB 92, in accordance with an application of the present invention.

FIGS. 3A-C, 3F-G, and 3I show elements of prosthetic aortic valve 20 prior to assembly of prosthetic aortic valve 20, and FIGS. 3D, 3E and 3H show these elements after assembly of prosthetic aortic valve 20. For clarity of illustration, FIG. 3H does not show leaflets 32, although they provided in practice.

In some of the configurations shown in FIGS. 3A-G and 3I (and FIGS. 1A-B), distal (e.g., upstream) ones 70 of interconnected stent cells 192 are located in a distal (e.g., upstream) half of frame 30 and define respective distal (e.g., upstream) peaks 72. At least one electrode 34, such as a cathode 54 (as labeled) or an anode 56 (configuration not labeled), is disposed at or near (e.g., within 8 mm of) a distal (e.g., upstream) peak 72 of one 74 of the distal (e.g., upstream) stent cells 70 (and is thus referred to herein as a distal (e.g., upstream) electrode 34). First and second distal (e.g., upstream) stent struts 76A and 76B of the one 74 of distal (e.g., upstream) stent cells 70 are joined at the distal (e.g., upstream) peak 72 (the distal (e.g., upstream) peak 72 is obscured in FIG. 3D, but can be seen in the adjacent stent cells). Optionally, such as shown, the distal (e.g., upstream) ones 70 of stent cells 192 are distal (e.g., upstream)-most ones of stent cells 192, and the one 74 of distal (e.g., upstream) stent cells 70 is one 74 of distal (e.g., upstream)-most stent cells 192. Alternatively, the distal (e.g., upstream) ones 70 of stent cells 192 are not distal (e.g., upstream)-most ones of stent cells 192, and the one 74 of distal (e.g., upstream) stent cells 70 is not one 74 of distal (e.g., upstream)-most stent cells 192, for example as shown in FIG. 3E.

In some of the configurations shown in FIGS. 3A-G and 3I (and FIGS. 1A-B), at least one electrode 34, such as an anode 56 (as labeled) or a cathode 54 (configuration not labeled), is disposed on a proximal (e.g., downstream) portion of frame 30, such as (a) a proximal (e.g., downstream) half of frame 30, (b) a portion of frame 30 proximal (e.g., downstream) of prosthetic leaflets 32, and/or (c) a portion of frame defined by a proximal-most (e.g., downstream-most) two rows of stent cells. This at least one electrode 34 is thus referred to herein as a proximal (e.g., downstream) electrode 34.

In some of the configurations shown in FIGS. 3A-G and 3I, prosthetic aortic valve 20 further comprises coupling material 80, which is shaped so as to define:
a first strip 82A that is mechanically coupled to first distal (e.g., upstream) stent strut 76A,
a second strip 82B that is mechanically coupled to second distal (e.g., upstream) stent strut 76B, and
a junction 84, which couples together first and second strips 82A and 82B, such that first and second strips 82A and 82B together couple electrode 34, such as a cathode 54, to frame 30 at or near (e.g., within 8 mm of) distal (e.g., upstream) peak 72. Using first and second strips 82A and 82B in this arrangement to couple electrode 34 to frame 30 typically helps stabilize electrode 34 with respect to frame 30, both during expansion of frame 30 from its compressed elongated state, and during many cardiac cycles after implantation of frame 30.

Optionally, first and second strips 82A and 82B are integrally joined at junction 84, e.g., integrally formed from a single piece of material (such as shown); alternatively, first and second strips 82A and 82B comprise discrete pieces of material coupled together at junction 84 (configuration not shown). First strip 82A may be mechanically coupled to either surface of first distal (e.g., upstream) stent strut 76A, and second strip 82B may be mechanically coupled to either surface of second distal (e.g., upstream) stent strut 76B.

For some applications, first and second strips 82A and 82B are mechanically coupled to first and second distal (e.g., upstream) stent struts 76A and 76B, respectively, by stitching, such as shown (to this end, first and second strips 82A and 82B may comprise stitching holes, as shown).

For some applications, junction 84 of coupling material 80 is mechanically coupled to frame 30 at or near (e.g., within 5 mm of) distal (e.g., upstream) peak 72.

For some applications:
first strip 82A has length equal to at least 50% of a length of first distal (e.g., upstream) stent strut 76A; for example, the length of first strip 82A may be greater than the length of first distal (e.g., upstream) stent strut 76A, such as at least 120% of the length of first distal (e.g., upstream) stent strut 76A (which may aid with mechanically coupling first strip 82A to first distal (e.g., upstream) stent strut 76A), and/or
second strip 82B has length equal to at least 50% of a length of second distal (e.g., upstream) stent strut 76B, such as least 75%, e.g., 100% of the length of second distal (e.g., upstream) stent strut 76B, and/or no more than 100% of the length of second distal (e.g., upstream) stent strut 76B.
For some applications, the one 74 of distal (e.g., upstream) stent cells 70 is a first one 74 of distal (e.g., upstream) stent cells 70, and the first one 74 of distal (e.g., upstream) stent cells 70 is joined at a cell junction 86 (node) to a circumferentially-adjacent second one 88 of distal (e.g., upstream) stent cells 70. Second strip 82B is mechanically coupled to cell junction 86, such as by stitching, such as shown (to this end, second strip 82B may comprise a stitching hole, as shown).

For some applications, prosthetic aortic valve 20 further comprises electrical lead 90 (shown schematically in the enlargement in FIG. 3A), which is electrically coupled to electrode 34 (and typically circuitry 40, if provided). First strip 82A is mechanically coupled to at least a portion of electrical lead 90.

For some of these applications, first strip 82A comprises electrical insulation, and first strip 82A electrically insulates the at least a portion of electrical lead 90 (such that first strip 82A and electrical lead 90 together provide an electrode lead). For some of these applications, first strip 82A comprises an elongate portion 91 of PCB 92 with which electrical lead 90 is integral (e.g., encased within PCB 92, such as by lamination, or disposed on an external surface of PCB 92 and coated with an electrically insulating coating). Typically, electrical lead 90 comprises a track (also known as a conductive trace) of PCB 92. In this configuration, PCB 92 typically also defines second strip 82B and junction 84 of coupling material 80. Although elongate portion 91 of PCB 92 is shown as oriented in a generally distal-proximal (e.g., upstream-downstream) orientation, elongate portion 91 of PCB 92 may also be at least partially oriented in a circumferential (angular) orientation around a portion of frame 30, such as shown in FIG. 3I, in which PCB 92 is shaped so as to define a generally distal-proximal (e.g., upstream-downstream) oriented elongate portion labeled 91, as well as a circumferentially-oriented (angularly-oriented) elongate portion oriented circumferentially (angularly) around a circumferential (angular) portion of frame 30 (horizontal in the figure).

Alternatively, first strip 82A is non-electrically-insulating, in which case electrical lead 90 may be electrically insulated by separate electrical insulation.

For some applications, first and second strips 82A and 82B are outer first and second strips 82A and 82B, which are mechanically coupled to radially outer (with respect to central longitudinal axis 60 of frame 30) sides of first and second distal (e.g., upstream) stent struts 76A and 76B, respectively. Coupling material 80 is shaped so as to further define:
an inner first strip 94A that is mechanically coupled to a radially inner side of first distal (e.g., upstream) stent strut 76A, and
an inner second strip 94B that is mechanically coupled to a radially inner side of second distal (e.g., upstream) stent strut 76B.

Junction 84 of coupling material 80 couples together outer first strip 82A, outer second strip 82B, inner first strip 94A, and inner second strip 94B. Outer first strip 82A, outer second strip 82B, inner first strip 94A, and inner second strip 94B together couple electrode 34 to frame 30 at or near distal (e.g., upstream) peak 72.

For some of these applications, junction 84 of coupling material 80 is folded over distal (e.g., upstream) peak 72, such as shown, such as shown in FIGS. 3C-D. Optionally, the folded junction 84 is mechanically coupled to frame 30 at or near distal (e.g., upstream) peak 72, such as by stitching, such as shown (to this end, junction 84 may comprise stitching holes 95, as shown). Prior to being folded over during assembly of prosthetic aortic valve 20, junction 84 may generally have an X-shape, such as shown in FIGS. 3A-B. A fold line 93 is schematically labeled in the enlargement of FIG. 3A.

Reference is still made to FIGS. 3A-D, and is again made to FIGS. 1A-B. For some applications, electrical lead 90, which electrically couples one or more electrodes 34 to circuitry 40, is integral with elongate portion 91 of PCB 92 (electrical lead 90 is shown schematically in the enlargement of FIG. 3A). As shown in FIGS. 1A-B and 3D, elongate portion 91 of PCB 92 is mechanically coupled to some of interconnected stent struts 190 of frame 30, such as by suturing using sutures 96. Elongate portion 91 of PCB 92 thus serves both to provide electrical insulation to electrical lead 90 and to facilitate coupling of electrical lead 90 to stent struts 190. This encasing of electrical lead 90 in elongate portion 91 of PCB 92 may be implemented either in combination with the techniques for mechanically coupling junction 84 to frame 30 at or near distal (e.g., upstream) peak 72 described above with reference to FIGS. 3A-D, or independently of these techniques.

As described above, for some applications, which electrical lead 90, which electrically couples one or more electrodes 34 to circuitry 40, is integral with elongate portion 91 of PCB 92. For some of these applications, the one or more electrodes 34 are mechanically coupled to frame 30:
downstream of prosthetic leaflets 32 and/or at a proximal (e.g., downstream) half of frame 30, such as shown for anode 56 in FIGS. 1A-B, and/or
upstream of the prosthetic leaflets 32, optionally at or near (e.g., within 8 mm of) respective distal (e.g., upstream) peaks 72 of respective ones 74 of distal (e.g., upstream)-most ones 70 of stent cells 192.

In any of these configurations, the one or more electrodes 34 may be directly coupled to frame 30, or may be indirectly coupled to frame 30 by being coupled to elongate portion 91 of PCB 92, which in turn is directly coupled to frame 30. In addition, in any of these configurations, circuitry 40 may be mechanically coupled to frame 30 proximal (e.g., downstream) of prosthetic leaflets 32.

For some applications, elongate portion 91 of PCB 92 has an undulating shape that generally runs along interconnected stent struts 190, such as shown in FIGS. 1A-B and 3D-E.

Alternatively or additionally, for some applications, elongate portion 91 of PCB 92 is shaped so as to follow a path of interconnected stent struts 190, such as shown in FIGS. 1A-B and 3D-E, and/or has a same general shape as interconnected stent struts 190, also as shown in FIGS. 1A-B and 3D-E.

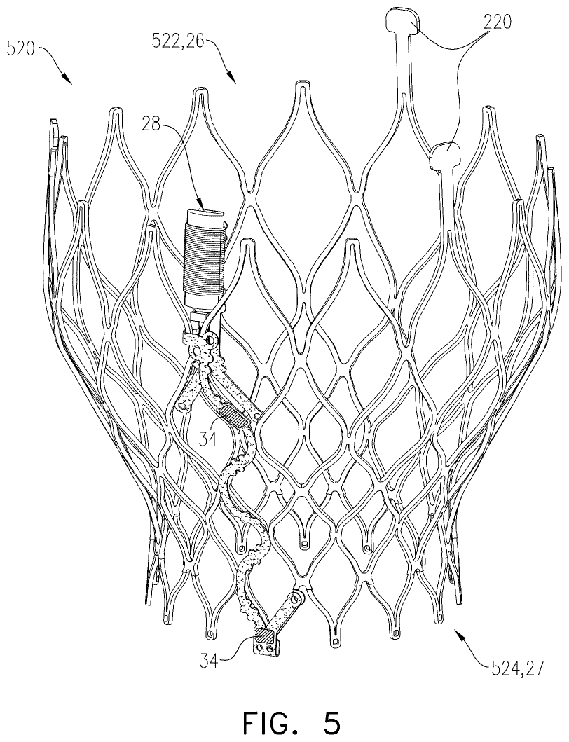

For some applications, elongate portion 91 of PCB 92 has one or more of the following lengths:
at least 50%, no more than 100%, and/or 50%-100% of a length of frame 30, measured parallel to central longitudinal axis 60 of frame 30 (labeled in FIGS. 1A-B),
at least 150%, no more than 1000%, and/or 150%-1000% of a greatest dimension of circuitry portion 100 of PCB 92 (in configurations in which circuitry portion 100 is elongate, the greatest dimension may equal the length of long lateral side 105 of circuitry portion 100, labeled in FIG. 3C), and/or
at least 0.5 cm, no more than 6 cm, and/or 0.5-6 cm, such as at least 0.5 cm, no more than 4 cm, and/or 0.5-4 cm (e.g., for prosthetic aortic valve 20), or at least 1.5 cm, no more than 6 cm, and/or 1.5-6 cm (e.g., for the mitral or tricuspid valve, such as described hereinbelow with reference to FIG. 5).

All of the above-mentioned lengths of elongate portion 91 are measured in a straight line between endpoints of elongate portion 91, even in configurations in which elongate portion 91 includes curved portions.

Alternatively or additionally, for some applications, a portion of elongate portion 91 between (a) circuitry portion 100 of PCB 92 and (b) a closest of the one or more electrodes 34 coupled by electrical lead 90 to circuitry 40 has one or more of the following lengths:
at least 50%, no more than 100%, and/or 50%-100% of a length of frame 30, measured parallel to central longitudinal axis 60 of frame 30 (labeled in FIGS. 1A-B),
at least 150%, no more than 1000%, and/or 150%-1000% of a greatest dimension of circuitry portion 100 of PCB 92 (in configurations in which circuitry portion 100 is elongate, the greatest dimension may equal the length long lateral side 105 of circuitry portion 100, labeled in FIG. 3C), and/or
at least 0.5 cm, no more than 6 cm, and/or 0.5-6 cm, such as at least 0.5 cm, no more than 4 cm, and/or 0.5-4 cm (e.g., for configurations in which the closest electrode 34 is disposed proximal (e.g., downstream) of prosthetic leaflets 32 and/or at a proximal (e.g., downstream) half of frame 30), or at least 1.5 cm, no more than 6 cm, and/or 1.5-6 cm (e.g., for configurations in which the closest electrode 34 is disposed distal (e.g., upstream) of the prosthetic leaflets 32).

All of the above-mentioned lengths of elongate portion 91 are measured in a straight line between endpoints of elongate portion 91, even in configurations in which elongate portion 91 includes curved portions.

For some applications, elongate portion 91 of PCB 92 has one or more of the following widths (perpendicular to a thickness of PCB 92):
at least 0.4, no more than 1.5, and/or 0.4-1.5 mm, and/or
at least 20%, no more than 120%, and/or 20%-120% of a shortest dimension of circuitry portion 100 of PCB 92 perpendicular to a thickness of circuitry portion 100 (in configurations in which circuitry portion 100 is elongate, the shortest dimension may be measured perpendicular to long lateral side 105 of circuitry portion 100, labeled in FIG. 3C).

As mentioned above, electrical lead 90 is coupled to electrode 34. For some applications, electrical lead 90 is coupled to cathode 54, while for other applications, electrical lead 90 is coupled to anode 56. Optionally, more than one electrical lead 90 is integral with elongate portion 91 of PCB 92, in which case a first one of electrical leads 90 may be coupled to cathode 54 and a second one of electrical leads 90 may be coupled to anode 56.

Optionally, a plurality of electrical leads 90 are integral with a corresponding plurality of elongate portions of PCB 92, such as described hereinbelow with reference to FIGS. 3F and 3G.

Optionally, one or more electrodes 34, e.g., one or more cathodes 54 and/or one or more anodes 56, are formed integrally with PCB 92.

Typically, both stent struts 190 and elongate portion 91 of PCB 92 are rectangular in cross section taken perpendicular to respective longitudinal axes of the stent struts and the elongate portion. Typically, electrical lead 90 is also rectangular in cross section, or trapezoidal in cross section. These rectangular cross sections enable flush coupling and/or good crimping of elongate portion 91 to stent struts 190.

For some applications:
stent struts 190 have a thickness of at least 150 microns, such as at least 300 microns; no more than 500 microns; and/or 150-500 microns, such as 300-500 microns,
stent struts 190 have a width of 200-700 microns,
a ratio of the width to the thickness of stent struts 190 is 0.5-2,
electrical lead 90 has a thickness of 5-80 microns, e.g., 50 microns,
electrical lead 90 has a width of 50-300 microns,
a ratio of the width to the thickness of electrical lead 90 is 5-50,
elongate portion 91 of PCB 92 has a thickness of at least 50 microns, no more than 150 microns, and/or 50-150 microns, and/or
elongate portion 91 of PCB 92 has a width of 300-1500 microns, and/or
a ratio of the width to the thickness of elongate portion 91 of PCB 92 is 3-20. Alternatively or additionally, for some applications:
a ratio of a thickness of stent struts 190 to a thickness of electrical lead 90 is at least 5, no more than 15, and/or 5-15, and/or
a ratio of a thickness of stent struts 190 to a thickness of elongate portion 91 of PCB 92 is at least 2, no more than 5, and/or 2-5.

Elongate portion 91A and/or bifurcation elongate portions 91B of PCB 92, described hereinbelow with reference to FIGS. 3F and/or 3G, may also have the dimensions provided immediately above for elongate portion 91 of PCB 92. Similarly, main portion 90A of electrical lead 90, bifurcation portions 90B of electrical lead 90, electrical lead 90C, electrical lead 90D, and/or electrical lead 90E, described hereinbelow with reference to FIGS. 3F and/or 3G, may also have the dimensions provided immediately above for electrical lead 90.

For some applications, as shown highly schematically in FIG. 3F, PCB 92 comprises a circuitry portion 100, such as an end portion 102 of PCB 92, distinct from elongate portion 91 of PCB 92, and circuitry 40 is coupled to circuitry portion 100 of PCB 92. For some applications, circuitry 40 further comprises (a) tracks 104 (also known as conductive traces) of PCB 92, (b) conductive pads of PCB 92, and (c) electronic components 106 coupled to PCB 92. Elongate portion 91 extends directly from circuitry portion 100 (e.g., end portion 102), and is typically integral with circuitry portion 100 (e.g., end portion 102). (In configurations in which circuitry portion 100 is a mid-portion of PCB 92, rather than end portion 102, PCB 92 extends beyond circuitry portion 100, such as to provide electrical connection to additional elements, e.g., one or more electrodes and/or additional circuitry.) Electrical lead 90 is typically integrally fabricated as a track of elongate portion 91 of PCB 92 in connection with one or more of tracks 104 of PCB 92 that are part of circuitry 40, which obviates the need for a separate connection point between electrical lead 90 and circuitry 40.

For some of these applications, antenna 28 is coupled to circuitry 40 by being coupled to one side of circuitry portion 100 of PCB 92, such as shown in FIGS. 1A-B. Optionally, elongate portion 91 of PCB 92 is shaped so as to define a plurality of protrusions 98 along elongate portion 91, which inhibit sutures 96 from sliding along elongate portion 91, such that the sutures 96 fix elongate portion 91 of PCB 92 securely to stent struts 190. Typically, protrusions 98 protrude laterally from elongate portion 91 of PCB 92 in a plane defined by PCB 92, either bidirectionally or in a single direction; optionally, some of protrusions 98 protrude bidirectionally and others of protrusions 98 protrude in a single direction, such as shown in the figures. Optionally, as labeled in the enlargement of FIG. 3A, an average distance D of lateral protrusion of protrusions 98 beyond non-protruding portions of elongate portion 91, in a single direction, equals 20%-100% of widths W of elongate portion 91 of PCB 92 at respective locations of the protrusions 98 along elongate portion 91, the average distance D and the widths W measured in the plane defined by PCB 92.

Reference is now made to FIGS. 3F and 3G. In these configurations, elongate portion 91 of PCB 92 is bifurcated, so as to define a main elongate portion 91A and two or more bifurcation elongate portions 91B. By way of example, exactly two bifurcation elongate portions 91B are shown in FIGS. 3F and 3G; in practice, elongate portion 91 may define more than two bifurcation elongate portions, such as three, four, five, six, or more bifurcation elongate portions.

In some applications, respective electrodes 34, e.g., respective cathodes 54, are coupled to respective bifurcation elongate portions 91B at a respective plurality of angular locations around frame 30. For some applications, such as shown in FIGS. 3F and 3G, respective electrodes 34 (e.g., respective cathodes 54) are disposed at a common axial position along central longitudinal axis 60 of frame 30 when prosthetic aortic valve 20 is in the expanded deployment configuration. For other applications (configuration not shown), respective electrodes 34 are disposed at different respective axial positions along central longitudinal axis 60 when prosthetic aortic valve 20 is in the expanded deployment configuration; for some of these other applications, respective electrodes 34 are disposed at a common angular location with respect to central longitudinal axis 60 when the prosthetic aortic valve is in the expanded deployment configuration.

In some applications, such as shown in FIG. 3F, an electrical lead 90 integral with elongate portion 91 of PCB 92 is bifurcated, so as to define a main portion 90A and two or more bifurcation portions 90B integral with respective bifurcation elongate portions 91B of elongate portion 91 of PCB 92. For example, each of the bifurcation portions 90B of electrical lead 90 may be electrically coupled to a respective electrode 34, e.g., a respective cathode 54, in which case these electrodes are in electrical communication with each other. A separate electrical lead 90C may be provided integral with main elongate portion 91A of elongate portion 91 of PCB 92, in electrical connection with another electrode 34, e.g., an anode 56.

In other applications, such as shown in FIG. 3G, at least two electrical leads 90D and 90E integral with elongate portion 91 of PCB 92. Electrical leads 90D and 90E are partially integral with main elongate portion 91A of elongate portion 91 of PCB 92, and partially integral with respective bifurcation elongate portions 91B of elongate portion 91 of PCB 92. For example, each of electrical leads 90 may be electrically coupled to a respective electrode 34, e.g., a respective cathode 54, in which case these electrodes (e.g., cathodes) are in electrically isolated from each other, and separately electrically connected to circuitry 40. A separate electrical lead 90C may be provided integral with main elongate portion 91A of elongate portion 91 of PCB 92, in electrical connection with another electrode 34, e.g., an anode 56.

For some applications, such as in the configurations described with reference to FIGS. 3F and 3G, circuitry 40 is configured to apply a pacing signal using all of electrodes 34, e.g., all of cathodes 54. For other applications, such as in the configuration described with reference to FIG. 3G, circuitry 40 is configured to apply the pacing signal using fewer than all of electrodes 34, e.g., (a) fewer than all of cathodes 54, for example, using just a single one of cathodes 54, or two or more cathodes 54 of three or more provided cathodes 54, and/or fewer than all of anodes 56, for example, using just a single one of anodes 56, or two or more anodes 56 of three or more provided anodes 56.

Optionally, in configurations in which prosthetic aortic valve 20 comprises a plurality of distal (e.g., upstream) electrodes 34, one or more of the distal (e.g., upstream) electrodes 34 are activated as one or more anodes 56, and one or more other distal (e.g., upstream) electrodes 34 are activated as one or more cathodes 54; in other words, any given distal (e.g., upstream) electrode 34 can be activated as either an anode 56 or a cathode 54.

Alternatively or additionally, optionally, in configurations in prosthetic aortic valve 20 comprises a plurality of proximal (e.g., downstream) electrodes 34, one or more of the proximal (e.g., downstream) electrodes 34 are activated as one or more anodes 56, and one or more other proximal (e.g., downstream) electrodes 34 are activated as one or more cathodes 54; in other words, any given proximal (e.g., downstream) electrode 34 can be activated as either an anode 56 or a cathode 54.

In general, any of electrodes 34 (regardless of their location on frame 30) can be configured as an anode 56 or a cathode 54.

For some applications, circuitry 40 separately activates each of electrodes 34, e.g., cathodes 54 and/or anodes 56, at different times in different combinations, and, based on a determination of which of the electrodes 34 (e.g., cathodes 54, and/or anodes 56 in configurations in which a plurality of anodes 56 are provided) provides the most effective pacing, i.e., the pacing that is successfully obtained using the smallest stimulation voltage. Circuitry 40 uses this most effective combination of electrodes 34, e.g., cathode(s) 54 or anode(s) 56, for future pacing.

For some applications, the determination regarding the most effective pacing is made based on the sensed ECG and/or EGM, as described hereinabove with reference to FIGS. 1A-B and 2, e.g., based on the combination of electrodes that results in the lowest ECG and/or EGM sensing threshold. Alternatively or additionally, for some applications, the determination regarding the most effective pacing is made by selecting the combination of electrodes that yields the lowest power, voltage, or current threshold sufficient for pacing, i.e., successful generation of a cardiac action potential.

In general, circuitry 40 is configured to apply the weakest pacing signal that yields an action potential in the heart. Circuitry 40 may be configured to induce pacing at a set voltage level or alternatively may be set to automatically determine the minimal voltage level of stimulation for a sufficient pacing.

For example, this determination regarding the most effective pacing may be made by circuitry 40 and/or by circuitry of an external control unit, such as external control unit 400, described hereinbelow with reference to FIG. 6. For some applications, this determination is performed (a) only once at the setup of the device immediately after implantation, (b) periodically, e.g., approximately once per day or once per week, and/or (c) before each pacing pulse is applied. An operator may or may not be involved in making the determination.

In some applications, this determination regarding the most effective pacing may be made by activating one or more of the distal (e.g., upstream) electrodes 34 as one or more anodes 56 (rather than as cathodes 54 as labeled in the drawings), and/or activating the distal (e.g., upstream) electrode 34 (or one or more of the distal (e.g., upstream) electrodes if a plurality are provided) as one or more cathodes 54 (rather than as one or more anodes 56 as labeled in the drawings).

Reference is still made to FIGS. 3F-G. It is noted that for clarity of illustration, electrical lead 90 (including main portion 90A and bifurcation portions 90B), electrical lead 90C, electrical lead 90D, and/or electrical lead 90E are shown highly schematically in FIGS. 3F-G. In practice, these electrical leads are typically rectangular in cross section, e.g., having the exemplary dimensions provided hereinabove with reference to FIGS. 1A-B and 3A-D. In addition, these electrical leads may be disposed running alongside one another, such as shown in FIGS. 3F-G, and/or in layers with PCB 92 (configuration not shown), as is known in the PCB art.

Reference is made to FIGS. 3C and 3H. In the configurations shown in these figures, elongate portion 91 extends directly from circuitry portion 100 (e.g., end portion 102), and is typically integral with circuitry portion 100 (e.g., end portion 102). An end portion 103 of elongate portion 91 is bent in a curve over at least a portion of circuitry portion 100, so as to sandwich one or more stent struts 190 between circuitry portion 100 and elongate portion 91, such as shown in FIG. 3H (although not shown in FIG. 3C for the sake of clarity, stent struts 190 are in fact present in prosthetic aortic valve 20). The configurations shown in FIGS. 3C and 3H may optionally be implemented in combination with the other configurations shown herein. (In practice, the one or more stent struts 190 are typically sandwiched more snugly between circuitry portion 100 and elongate portion 91 than shown in FIG. 3H.)

Typically, circuitry portion 100 is disposed radially inward from stent struts 190, end portion 103 of elongate portion 91 is bent in a curve over at least a portion of circuitry portion 100, and the non-curved portion of elongate portion 91 that extends distal (e.g., upstream) from end portion 103 is disposed radially outward from stent struts 190.

For some applications, circuitry portion 100 is elongate, and end portion 103 of elongate portion 91 extends from a long lateral side 105 of circuitry portion 100, such as shown in FIG. 3C, or from a proximal (e.g., downstream) end 107 of circuitry portion 100, such as shown in FIG. 3H. By contrast, if end portion 103 of elongate portion 91 were to instead extend from a distal (e.g., upstream) end of circuitry portion 100, elongate portion 91 might be more likely to be cut during crimping of frame 30. The circumferential width of stent cells 192 diminishes during crimping, while the height of stent cells 192 (in the axial direction) extends during crimping. If the rectangularly cross-sectioned elongate portion 91 were to cross the frame wall when extending from the distal (e.g., upstream) end of circuitry portion 100, elongate portion 91 might be squeezed between two struts during crimping, because the width of elongate portion 91 might be greater than the minimal distance between adjacent nodes or struts during crimping.

Reference is made to FIG. 3I. In this configuration, PCB 92 is shaped so as to define two or more circuitry portions 100 including a first circuitry portion 100A and a second circuitry portion 100B, for example, exactly two circuitry portions 100 (as shown) or three or more circuitry portions 100 (configuration not shown). One or more elongate circuitry-connecting portions 110 of PCB 92 connect the two or more circuitry portions 100. Typically, each of the one or more elongate circuitry-connecting portions 110 comprises one or more electrical leads that are integral with the respective elongate circuitry-connecting portion 110. For some applications, the one or more elongate circuitry-connecting portions 110 extend circumferentially around at least a portion of frame 30.

For some applications, the one or more elongate circuitry-connecting portions 110 are mechanically coupled to some of interconnected stent struts 190 of frame 30, and typically generally run along these stent struts (such that the one or more elongate circuitry-connecting portions 110 may have a zig-zag shape, for example).

Optionally, one of the two or more circuitry portions 100 (e.g., second circuitry portion 100B, as shown) is end portion 102 of PCB 92.

For some applications, circuitry 40 is distributed among the two or more circuitry portions 100, i.e., the two or more circuitry portions 100 comprises respective portions of electronic components of circuitry 40. This may allow the accommodation of circuitry 40 is case a single circuitry portion 100 does not have a sufficient surface area. For some applications, prosthetic aortic valve 20 comprises an energy storage module, e.g., comprising a battery, which is coupled to one of circuitry portions 100. As used in the present application, including in the claims, “circuitry” means a combination of (a) one or more electronic components 106 and (b) one or more tracks 104 (also known as conductive traces) of a PCB electrically coupled to the one or more electrically components, typically by conductive pads of the PCB. The circuitry may or may not comprise a source of power. The one or more electronic components can be active components (e.g., semiconductor devices, such as integrated circuits, transistors, and/or active diodes); passive components (e.g., electrodes, capacitors, and/or passive diodes); and/or energy storage modules (e.g., comprising a battery). As used in the present application, including in the claims, tracks (also known as traces), electrical leads, wires, and cables are not considered to be electronic components.

Reference is again made to FIGS. 1A-B. The following configuration may be implemented alone or in combination with any of the other configurations described herein, including hereinabove with reference to FIGS. 3A-G and 3I. In this configuration, interconnected stent cells 192 of interconnected stent struts 190 of frame 30 include a first stent cell 170 shaped so as to define:
two peaks 172, consisting of a distal (e.g., upstream) peak 172A and a proximal (e.g., downstream) peak 172B,
two lateral nodes 186, consisting of a left lateral node 186A and a right lateral node 186B,
two left stent struts 176, consisting of (a) a distal (e.g., upstream) left stent strut 176A joined with distal (e.g., upstream) peak 172A and left lateral node 186A, and (b) a proximal (e.g., downstream) left stent strut 176B joined with proximal (e.g., downstream) peak 172B and left lateral node 186A, and
two right stent struts 178, consisting of (a) a distal (e.g., upstream) right stent strut 178A joined with distal (e.g., upstream) peak 172A and right lateral node 186B, and (b) a proximal (e.g., downstream) right stent strut 178B joined with proximal (e.g., downstream) peak 172B and right lateral node 186B.

For example, first stent cell 170 may be located in a proximal (e.g., downstream) half of frame 30, such as shown, e.g., first stent cell 170 may be a proximal (e.g., downstream)-most stent cell (configuration not shown). Alternatively, first stent cell 170 may be located in a distal (e.g., upstream) half of frame 30 (configuration not shown in FIGS. 1A-B), e.g., first stent cell 170 may be a distal (e.g., upstream)-most stent cell (configuration not shown in FIGS. 1A-B).

In this configuration, prosthetic aortic valve 20 comprises an electronic component 150, which is disposed at or near one of peaks 172. For example, electronic component 150 may be part of circuitry 40 (such as shown), may comprise antenna 28 (also such as shown), may comprise an energy storage module, e.g., comprising a battery, or may comprise an electrode 34.

In this configuration, prosthetic aortic valve 20 further comprises coupling material 180, which is shaped so as to define:
a first strip 182A that is mechanically coupled to at least one of left stent struts 176,
a second strip 182B that is mechanically coupled to at least one of right stent struts 178, and
a junction 184, which couples together the first and the second strips 182A and 182B,
   such that first and second strips 182A and 182B together couple electronic component 150 to frame 30 at or near (e.g., within 15 mm of) the one of peaks 172. Using first and second strips 182A and 182B in this arrangement to couple electronic component 150 to frame 30 typically helps stabilize electronic component 150 with respect to frame 30, both during expansion of frame 30 from its compressed elongated state, and during many cardiac cycles after implantation of frame 30.

By way of example and not limitation, in FIGS. 1A-B, first strip 182A is shown mechanically coupled to proximal (e.g., downstream) left stent strut 176B, and second strip 182B is shown mechanically coupled to proximal (e.g., downstream) right stent strut 178B, such that first and second strips 182A and 182B together couple electronic component 150 to frame 30 at or near proximal (e.g., downstream) peak 172B. Alternatively, first strip 182A may be mechanically coupled to distal (e.g., upstream) left stent strut 176A, and second strip 182B may be mechanically coupled to distal (e.g., upstream) right stent strut 178A, such that first and second strips 182A and 182B together couple electronic component 150 to frame 30 at or near distal (e.g., upstream) peak 172A (configuration not shown).

Optionally, first and second strips 182A and 182B are integrally joined at junction 184, e.g., integrally formed from a single piece of material (such as shown); alternatively, first and second strips 182A and 182B comprise discrete pieces of material coupled together at junction 184 (configuration not shown). First strip 182A may be mechanically coupled to either surface of the at least one of left stent struts 176, and second strip 182B may be mechanically coupled to either surface of the at least one of right stent struts 178.

For some applications, first and second strips 182A and 182B together couple electronic component 150 to frame 30 at least partially outside the first stent cell at or near the one of peaks 172.

For some applications, first and second strips 182A and 182B are mechanically coupled to the at least one of left stent struts 176 and the at least one of right stent struts 178, respectively, by stitching.

For some applications, junction 184 of coupling material 180 is mechanically coupled to frame 30 at or near the one of peaks 172, such as by stitching.

For some applications, first strip 182A has length equal to at least 50% of a length of the at least one of left stent struts 176; for example, the length of first strip 182A may be greater than the length of the at least one of left stent struts 176. Alternatively or additionally, for some applications, second strip 182B has length equal to at least 50% of a length of the at least one of right stent struts 178; for example, the length of second strip 182B may be greater than the length of the at least one of right stent struts 178.

For some applications, first strip 182A is mechanically coupled to left lateral node 186A, such as by stitching. Alternatively or additionally, for some applications, second strip 182B is mechanically coupled to right lateral node 186B, such as by stitching.

For some applications, prosthetic aortic valve 20 further comprises an electrical lead, such as electrical lead 90, which is electrically coupled to electronic component 150, and first strip 182A is mechanically coupled to at least a portion of the electrical lead. For some of these applications, first strip 182A comprises electrical insulation, and first strip 182A electrically insulates the at least a portion of the electrical lead. For some applications, first strip 182A comprises an elongate portion of a PCB with which the electrical lead is integral, such as elongate portion 91 of PCB 92.

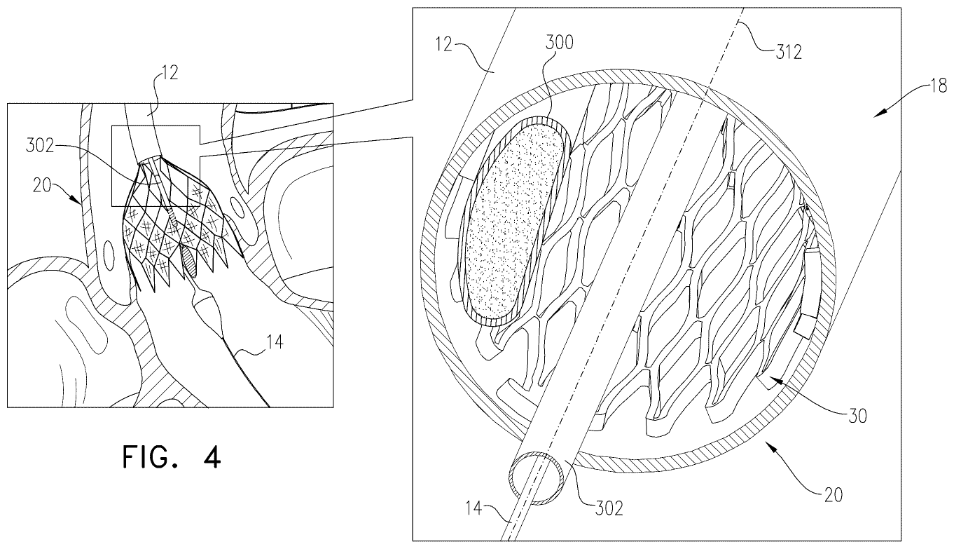

In some applications of the present invention, the prosthetic valves described herein implement some or all of the features described in PCT Publication WO 2025/041129 to Gross et al., with reference to FIGS. 4, 5A-B, 6A-B, 7, 8A-B, and/or 10 thereof, mutatis mutandis.

Reference is now made to FIG. 4, which is a schematic illustration of a portion of prosthetic aortic valve 20 in a constrained delivery configuration within delivery sheath 12, in accordance with an application of the present invention.

Reference is now made to FIG. 5, which is a schematic illustration of a prosthetic atrioventricular valve 520, in accordance with an application of the present invention. Other than as described hereinbelow, prosthetic atrioventricular valve 520 may be generally similar to prosthetic aortic valve 20, described hereinabove with reference to FIGS. 1A-3I, and may implement any of the features thereof, mutatis mutandis. Prosthetic atrioventricular valve 520 may also optionally implement any of the features described hereinabove with reference to FIG. 4, mutatis mutandis, and/or described in PCT Publication WO 2025/041129 to Gross et al., with reference to FIGS. 4-8B thereof, mutatis mutandis.

Prosthetic atrioventricular valve 520 may, for example, be a prosthetic mitral valve or a prosthetic tricuspid valve. Typically, but not necessarily, prosthetic atrioventricular valve 520 has a shorter length than the configurations of prosthetic aortic valve 20 shown in the figures. Optionally, prosthetic atrioventricular valve 520 implements any techniques known in the art for transcatheter prosthetic atrioventricular valves. For example, prosthetic atrioventricular valve 520 may implement techniques described in PCT Publication WO 2022/118316 to Albitov et al., U.S. Pat. No. 11,246,704 to Hariton et al., US Patent Application Publication 2015/0328000 to Ratz et al., PCT Publication WO 2024/010739 to Garete et al., U.S. Pat. No. 10,973,628 to Levi, U.S. Pat. No. 10,299,927 to McLean et al., U.S. Pat. No. 10,828,153 to Noe et al., and/or U.S. Pat. No. 7,510,575 to Spenser et al., all of which are incorporated herein by reference.

Prosthetic atrioventricular valve 520 comprises a frame 530 and a plurality of prosthetic leaflets coupled to frame 530 so as to allow blood flow in a downstream direction and inhibit blood flow in an upstream direction when prosthetic atrioventricular valve 520 is in an expanded deployment configuration, such as shown in FIG. 5. For clarity of illustration, the prosthetic leaflets are not shown in FIG. 5; in practice they are provided, and may be similar to prosthetic leaflets 32, described hereinabove, mutatis mutandis, and/or may implement any techniques of prosthetic leaflets of prosthetic atrioventricular valves known in art, including, but not limited to, the techniques described in the patent publications incorporated hereinabove.

Prosthetic atrioventricular valve 520 has an upstream end 522 and a downstream end 524. Upstream end 522 may also be a proximal end 26 and downstream end 524 may also be a distal end 27, for example because proximal end 26 may be disposed in distal end portion 31 of delivery sheath 12 (described hereinabove with reference to FIG. 2) more proximally than distal end 27; in other words, proximal end 26 is closer to proximal end portion 29 of delivery sheath 12 than is distal end 27. For some applications, such as shown, proximal end 26 is configured to be coupled to delivery system 18 (e.g., shaped so as to define delivery-tool-coupling tabs 220, which are configured to removably couple frame 30, and thus prosthetic atrioventricular valve 520, to delivery system 18, e.g., to a delivery shaft of delivery system 18, such as described herein). For other applications (configuration not shown), distal end 27 is configured to be coupled to delivery system 18, such as to a capsule of the distal end portion 31 of delivery sheath 12, such as described hereinabove.

In some applications of the present invention, prosthetic atrioventricular valve 520 implements any of the techniques described hereinabove with reference to FIGS. 1A-4 for prosthetic aortic valve 20. In some of these techniques, “proximal” features of prosthetic aortic valve 20 are described as “downstream” features, and “distal” features of prosthetic aortic valve 20 are described as “upstream” features. Typically, in prosthetic atrioventricular valve 520 these directions are the opposite, such that “proximal” features of atrioventricular valve 520 are “upstream” features, and “distal” features of atrioventricular valve 520 are “downstream” features.

Reference is now made to FIG. 6, which is a schematic illustration of an external control unit 400 of valve prosthesis system 10, in accordance with an application of the present invention. A prosthetic cardiac valve, such as prosthetic aortic valve 20, prosthetic atrioventricular valve 520, prosthetic atrioventricular valve 720, prosthetic atrioventricular valve 820, or prosthetic atrioventricular valve 920, is configured to be delivered to a native cardiac valve of a patient in a constrained delivery configuration within delivery sheath 12 using guidewire 14, such as described hereinabove with reference to FIG. 2.

External control unit 400 is configured to be disposed outside a body of the patient, and comprises:
a housing 410, which is shaped so as to define a guidewire-receiving channel 412;
a rapid-pacing user control 414; and
external-unit control circuitry 418.

Reference is again made to FIG. 2. Typically, an external system is provided that is configured to be disposed outside a body of the patient. The external system comprises an external control unit 700, which may, for example, comprise external control unit 400, described hereinabove with reference to FIG. 6.

For some applications, the external system further comprises an external transmitter and/or receiver, which optionally comprises an external coil 420, which is highly schematically illustrated in FIG. 2. For example, external coil 420 may be configured to be placed around the patient's chest, such as schematically shown in FIG. 2, or placed against the chest without surrounding the chest, such as against the sternum (configuration not shown). The external transmitter and/or receiver is configured to wirelessly transfer energy to at least one of the one or more prosthetic-valve coils 36, such as by driving external coil 420 to wirelessly transfer the energy to at least one of the one or more prosthetic-valve coils 36 by inductive coupling. For example, the external transmitter may transmit RF energy at a frequency of 2-300 MHz, e.g., 6.78 MHz.

Reference is again made to FIG. 6. As described hereinabove with reference to FIGS. 1A-B and 2, for some applications, circuitry 40 is configured to apply both regular pacing and rapid pacing. For example, the rapid pacing may be applied during an invasive structural heart procedure, such as an implantation procedure, such as a TAVR-in-TAVR procedure in which the first TAVR comprises prosthetic aortic valve 20, and a portion of the regular pacing may be applied temporarily while the patient is hospitalized after implantation of prosthetic aortic valve 20. External control unit 400 may be provided for controlling both the regular pacing and the rapid pacing. (When the patient is discharged from the hospital, an external control unit is typically provided having fewer or no user controls accessible by the patient.) Because the user or healthcare works may have access to external control unit 400, it is desirable to prevent accidental activation of rapid pacing after completion of the implantation procedure.

For some applications, external-unit control circuitry 418 is configured to:
drive an external transmitter to wirelessly transfer energy to at least one of the one or more prosthetic-valve coils 36, such as for powering regular pacing (for example, by driving an energy-transmission coil of the external transmitter to wirelessly transfer the energy by inductive coupling), and
only upon activation of rapid-pacing user control 414 and when guidewire 14 is disposed within guidewire-receiving channel 412 of housing 410, drive the prosthetic aortic valve to apply rapid pacing using cathode 54 and anode 56.

To this end, external control unit 400 comprises a sensor, configured to sense whether guidewire 14 is disposed within guidewire-receiving channel 412 of housing 410.

This feature may serve as a safety feature, which restricts application of the rapid pacing to a transcatheter or surgical cardiovascular operation by a certified medical interventionalist.

For some applications, external-unit control circuitry 418 is configured to drive the one or more electrodes 34 to apply pacing; in this configuration, circuitry 40 of prosthetic aortic valve 20, if even provided, is generally passive, i.e., external-unit control circuitry 418 sets the parameters of the pacing signal. For example, in this configuration, circuitry 40 may comprise only passive electrical components, e.g., electrodes, capacitors, and/or passive diodes. Optionally, these techniques are implemented in combination with passive circuitry techniques described in U.S. Pat. No. 11,291,844 to Gross and/or PCT Publication WO 2022/149130 to Gross, both of which are incorporated herein by reference.

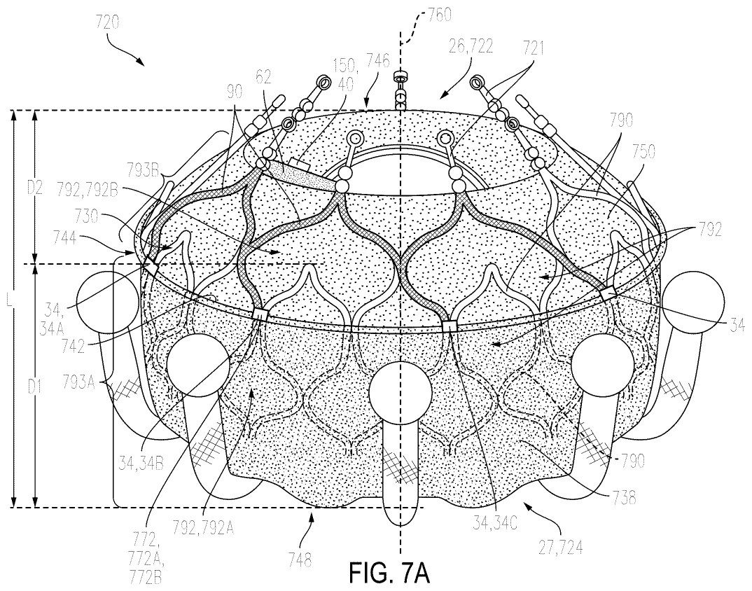

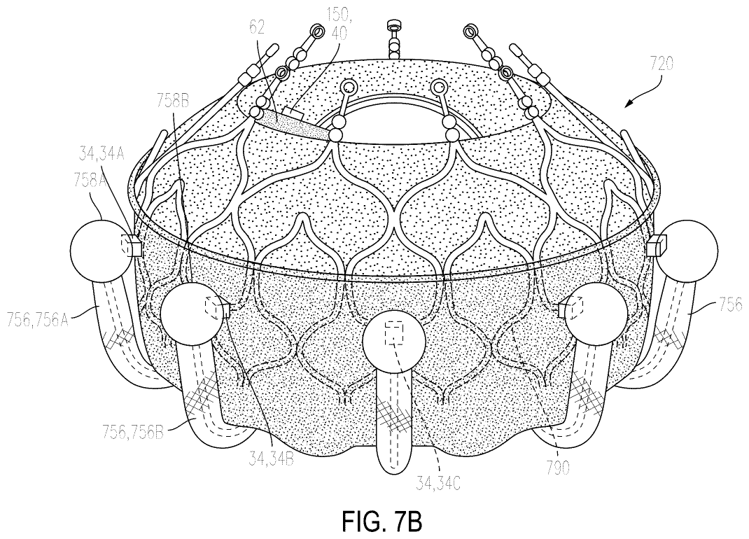

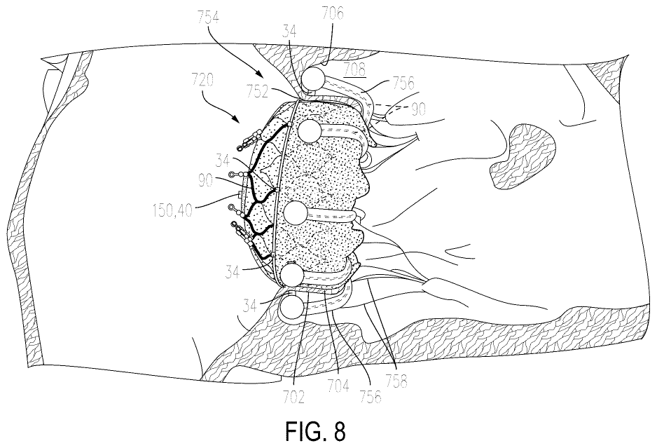

Reference is now made to FIGS. 7A-B and 8, which are schematic illustrations of a prosthetic atrioventricular valve 720, in accordance with respective applications of the present invention. FIGS. 7A-B show two respective configurations of prosthetic atrioventricular valve 720, and FIG. 8 shows another configuration that combines the features of the two respective configurations of FIGS. 7A-B. FIG. 8 also shows prosthetic atrioventricular valve 720 implanted in a native atrioventricular valve 754 (by way of example and not limitation, a native tricuspid valve). Other than as described hereinbelow, prosthetic atrioventricular valve 720 may be generally similar to prosthetic aortic valve 20, described hereinabove with reference to FIGS. 1A-3I; and/or prosthetic atrioventricular valve 520, described hereinabove with reference to FIG. 5, and may implement any of the features of either or both of these valves, mutatis mutandis. Prosthetic atrioventricular valve 720 may also optionally implement any of the features described hereinabove with reference to FIG. 4, mutatis mutandis, and/or described in PCT Publication WO 2025/041129 to Gross et al., with reference to FIGS. 4-8B thereof, mutatis mutandis. Like reference numerals refer to like elements.

Prosthetic atrioventricular valve 720 may, for example, be a prosthetic tricuspid valve or a prosthetic mitral valve. Optionally, prosthetic atrioventricular valve 720 implements any techniques known in the art for transcatheter prosthetic atrioventricular valves. For example, prosthetic atrioventricular valve 720 may implement techniques described in PCT Publication WO 2024/010739 to Garete et al. and/or in PCT Publication WO 2022/118316 to Albitov et al., U.S. Pat. No. 11,246,704 to Hariton et al., US Patent Application Publication 2015/0328000 to Ratz et al., U.S. Pat. No. 10,973,628 to Levi, U.S. Pat. No. 10,299,927 to McLean et al., U.S. Pat. No. 10,828,153 to Noe et al., and/or U.S. Pat. No. 7,510,575 to Spenser et al., all of which are incorporated herein by reference.

Prosthetic atrioventricular valve 720 comprises a frame 730; electrodes 34, including first and second electrodes 34A and 34B; and a plurality of prosthetic leaflets coupled to frame 730 so as to allow blood flow in a downstream direction and inhibit blood flow in an upstream direction when prosthetic atrioventricular valve 720 is in an expanded deployment configuration, such as shown in FIGS. 7A-B and 8. For clarity of illustration, the prosthetic leaflets are not shown in FIGS. 7A-B and 8; in practice they are provided, and may be similar to prosthetic leaflets 32, described hereinabove, mutatis mutandis, and/or may implement any techniques of prosthetic leaflets of prosthetic atrioventricular valves known in art, including, but not limited to, the techniques described in the patent publications incorporated hereinabove.

Frame 730 defines a central longitudinal axis 760 when prosthetic atrioventricular valve 720 is in the expanded deployment configuration. Frame 730 comprises interconnected stent struts 790 arranged so as to define interconnected stent cells 792.

Prosthetic atrioventricular valve 720 has an upstream end 722 and a downstream end 724. Upstream end 722 may also be a proximal end 26 and downstream end 724 may also be a distal end 27, for example because proximal end 26 may be disposed in distal end portion 31 of delivery sheath 12 more proximally than distal end 27; in other words, proximal end 26 is closer to proximal end portion 29 of delivery sheath 12 than is distal end 27. For some applications, such as shown, proximal end 26 is configured to be coupled to delivery system 18 (e.g., shaped so as to define delivery-tool-coupling tabs 721, which are configured to removably couple frame 30, and thus prosthetic atrioventricular valve 720, to delivery system 18, e.g., to a delivery shaft of delivery system 18, such as described herein). Optionally, delivery-tool-coupling tabs extend axially beyond interconnected stent cells 792. For other applications (configuration not shown), distal end 27 is configured to be coupled to delivery system 18, such as to a capsule of the distal end portion 31 of delivery sheath 12, such as described hereinabove.

In some applications of the present invention, prosthetic atrioventricular valve 720 implements any of the techniques described hereinabove with reference to FIGS. 1A-4 for prosthetic aortic valve 20. In some of these techniques, “proximal” features of prosthetic aortic valve 20 are described as “downstream” features, and “distal” features of prosthetic aortic valve 20 are described as “upstream” features. Typically, in prosthetic atrioventricular valve 720 these directions are the opposite, such that “proximal” features of atrioventricular valve 720 are “upstream” features, and “distal” features of atrioventricular valve 720 are “downstream” features.

First and second electrodes 34A and 34B are disposed at respective axial positions along central longitudinal axis 760 when prosthetic atrioventricular valve 720 is in the expanded deployment configuration, the axial positions the same or differing from one another. Each of the axial positions is at a downstream-end distance D1 from a downstream end 748 of stent cells 792 and is at an upstream-end distance D2 from an upstream end 746 of stent cells 792.

For some applications, the downstream-end distance D1 equals at least 20% (e.g., at least 25%, such as at least 30%) of a total axial length L of stent cells 792 measured between downstream end 748 and upstream end 746 of stent cells 792, and/or the upstream-end distance D2 equals at least 20% (e.g., at least 25%, such as at least 30%) of the total axial length L of stent cells 792. The downstream-end distance D1, the upstream-end distance D2, and the total axial length L of stent cells 792 are measured parallel to central longitudinal axis 760. Any of the electrodes of any of the prosthetic valves described herein may optionally implement this electrode positioning, mutatis mutandis.

Reference is made to FIGS. 7A and 8. For some applications, prosthetic atrioventricular valve 720 further comprises an outer skirt 738. A portion of stent cells 792 is coupled to a radially inner surface of outer skirt 738 so that:
outer skirt 738 covers (from outside) a first axial portion 793A of stent cells 792 and does not cover a second axial portion 793B of stent cells 792, and
an edge 742 of outer skirt 738 is disposed around a circumference of stent cells 792 axially at a border 744 between first and second axial portions 793A and 793B of stent cells 792.

For some applications, first electrode 34A is disposed at least partially on a radially outer surface of outer skirt 738, within 8 mm of edge 742 of outer skirt 738 when prosthetic atrioventricular valve 720 is in the expanded deployment configuration, such as within 5 mm, 4 mm, 3 mm, 3 mm, or 1 mm of edge 742.

For some applications, such as shown, first axial portion 793A of stent cells 792 is downstream of first axial portion 793A of stent cells 792. Alternatively, first axial portion 793A of stent cells 792 is upstream of first axial portion 793 A of stent cells 792 (configuration not shown).

For some applications, outer skirt 738 extends to or beyond either upstream end 746 or downstream end 748 of interconnected stent cells 792.

For some applications, prosthetic atrioventricular valve 720 further comprises circuitry 40 and electrical leads 90. The electrical leads include first and second electrical leads 90, which electrically couple first and second electrodes 34A and 34B, respectively, to circuitry 40. For some of these applications, first electrode 34A is coupled to interconnected stent struts 790 of first axial portion 793A of stent cells 792, and is not coupled to interconnected stent struts 790 of first axial portion 793A of stent cells 792. Optionally, the first electrical lead 90 runs along a portion of interconnected stent struts 790 to which the first electrical lead 90 is coupled. (By way of example and not limitation, FIGS. 7A and 8 show elongate portions of PCB 92 in which electrical leads 90 are encased, but the electrical leads 90 (wires) themselves are not visible, such as described hereinabove with reference to FIGS. 3A-G and I, and more particularly with reference to FIGS. 3F-G. Alternatively, other configurations of electrical leads are used, which are not integrated with elongate PCB.)

For some applications, prosthetic atrioventricular valve 720 further comprises an inner skirt 750. For some of these applications, at least a portion of stent cells 792 is coupled to a radially outer surface of inner skirt 750 so that inner skirt 750 at least partially covers (from inside) first axial portion 793A of stent cells 792. Typically, the first electrical lead 90 is coupled to at least a portion of interconnected stent struts 790 covered by inner skirt 750. As used in the present application, including in the claims, “covers” means covers from inside, outside, or a combination of inside and outside, unless otherwise specified. Optionally, prosthetic leaflets 32 are coupled to frame 730 via being coupled to inner skirt 750.

For some applications, second electrode 34B is disposed at least partially on the radially outer surface of outer skirt 738, within 8 mm of edge 742 of outer skirt 738 when prosthetic atrioventricular valve 720 is in the expanded deployment configuration, such as within 5 mm, 4 mm, 3 mm, 3 mm, or 1 mm of edge 742.

For some applications, such as shown in FIGS. 7A and 8, first and second electrodes 34A and 34B are disposed at a common axial position along central longitudinal axis 760 when prosthetic atrioventricular valve 720 is in the expanded deployment configuration.

For some of these applications, first and second electrodes 34A and 34B are disposed at or near respective peaks of first and second ones of stent cells 792, respectively, for example, at or near respective peaks 772 (which are both upstream peaks 772A of stent cells 792A and downstream peaks 772B of stent cells 792B). Alternatively or additionally, for some applications, first and second electrodes 34A and 34B are coupled to respective interconnected stent struts 790 of first and second ones of stent cells 792, respectively. Still further alternatively or additionally, for some applications, first and second electrodes 34A and 34B are coupled to frame 730 by being coupled to (optionally by coating) outer skirt 738 or another sheet.

For some applications, prosthetic atrioventricular valve 720 further comprises a third electrode 34C, which is disposed at least partially on the radially outer surface of outer skirt 738, within 8 mm of edge 742 of outer skirt 738 disposed at an axial position along central longitudinal axis 760 when prosthetic atrioventricular valve 720 is in the expanded deployment configuration, such as within 5 mm, 4 mm, 3 mm, 3 mm, or 1 mm of edge 742.

For some of these applications, first, second, and third electrodes 34A, 34B, and 34° C. (and, optionally, additional electrodes 34) are disposed at a common axial position along central longitudinal axis 760 when prosthetic atrioventricular valve 720 is in the expanded deployment configuration.

For some applications, frame 730 is shaped such that when prosthetic atrioventricular valve 720 is in the expanded deployment configuration after deployment at a native cardiac valve, such as shown in FIG. 8, first and second electrodes 34A and 34B are disposed at a height of an annulus 752 of native atrioventricular valve 754.

Reference is again made to FIGS. 7A-B and 8. In some applications, prosthetic atrioventricular valve 720 further comprises a plurality of anchoring arms 756, which are typically coupled to or integral with frame 730 (e.g., defined by struts of frame 730). When prosthetic atrioventricular valve 720 is in the expanded deployment configuration, anchoring arms 756 extend in a radially outward and upstream direction, so as to engage one or more portions of the heart and thereby help hold prosthetic atrioventricular valve 720 in place upon deployment, the one or more portions selected from the group consisting of: chordae tendineae 758 of the heart, and downstream surfaces 702 of leaflets 704 of native atrioventricular valve 754, such as shown in FIG. 8 for the tricuspid valve. Alternatively, prosthetic atrioventricular valve 720 may be implanted in the native mitral valve, mutatis mutandis, as described herein.

Reference is made to FIGS. 7B and 8. In some applications, first and second electrodes 34A and 34B are coupled to first and second ones 756A and 756B of anchoring arms 756, respectively. Optionally, additional electrodes 34, such as third electrode 34C, are coupled to additional ones of anchoring arms 756, respectively. As shown in FIG. 8, this electrode positioning may optionally be implemented with the electrode positioning described hereinabove with reference to FIG. 7A.

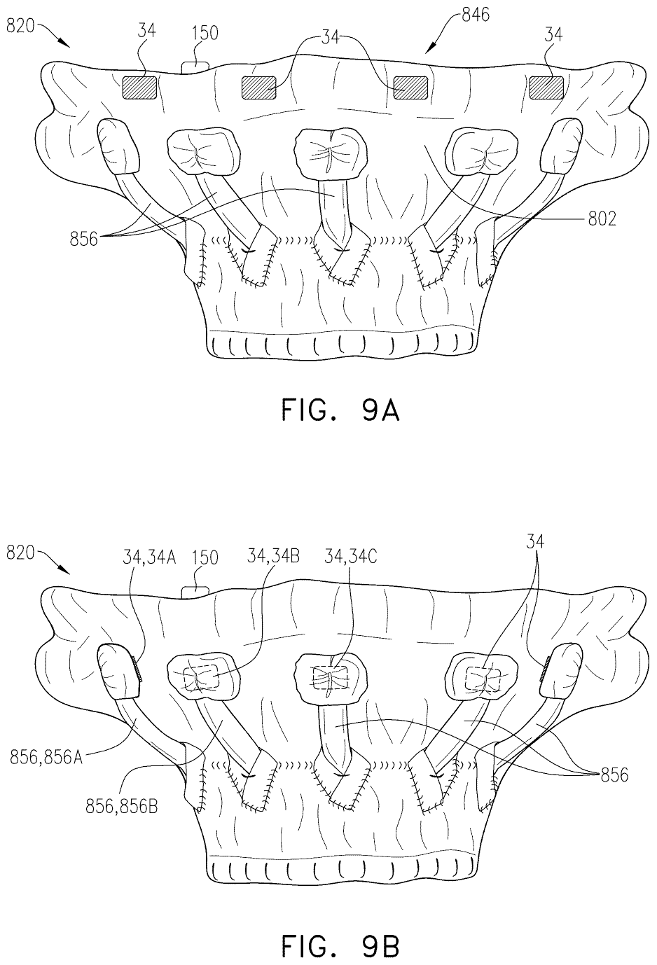

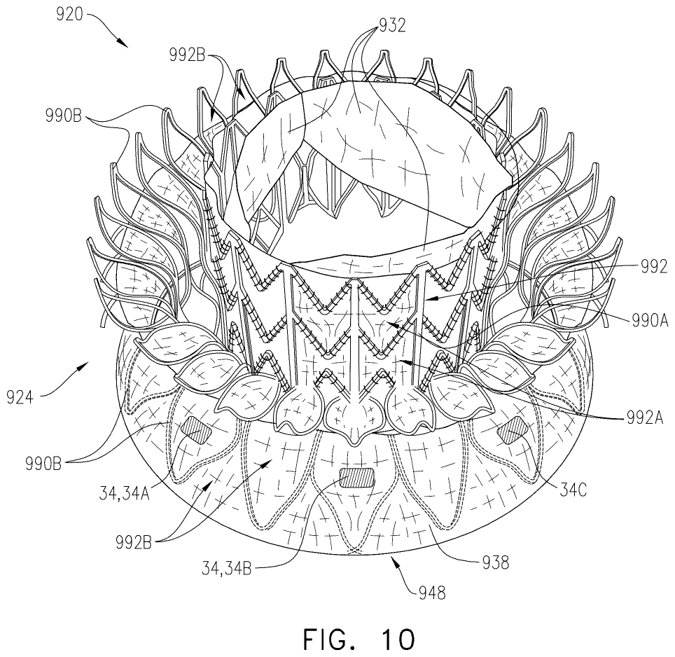

For some applications, first and second electrodes 34A and 34B are coupled to first and second anchoring arms 756A and 756B, respectively, at or within 5 mm (e.g., within 3 mm, within 2 mm, or within 1 mm) of respective free ends 758A and 758B of first and second anchoring arms 756A and 756B, respectively. Optionally, end portions of the free ends of the anchoring arms may be generally spherical, as shown in FIGS. 7A-B and 8, or may have another shape, such as the shape shown in prosthetic atrioventricular valve 820, as described hereinbelow with reference to FIGS. 9A-B.

For some applications, anchoring arms 756 are configured, when prosthetic atrioventricular valve 720 is in the expanded deployment configuration, to bring first and second electrodes 34A and 34B into contact with one or more surfaces selected from the group consisting of: the one or more downstream surfaces 702 of the one or more respective leaflets 704 of native atrioventricular valve 754, and a ventricular wall 706 in a subvalvular space 708 of native atrioventricular valve 754. Alternatively, one or more portions of prosthetic atrioventricular valve 720 other than anchoring arms 756 are configured, when prosthetic atrioventricular valve 720 is in the expanded deployment configuration, to bring first and second electrodes 34A and 34B into the contact with the one or more surfaces mentioned above; in this configuration, prosthetic atrioventricular valve 720 does not necessarily comprise anchoring arms 756; for example, a downstream portion of frame 730 may be shaped to bring the electrodes into this tissue contact when prosthetic atrioventricular valve 720 is in the expanded deployment configuration; other portions of prosthetic atrioventricular valve 720 may also be configured to bring the electrodes into this tissue contact.

Reference is again made to FIGS. 7A-B and 8. For some applications, circuitry 40 is configured to use one of first and second electrodes 34A and 34B as an anode and the other of first and second electrodes 34A and 34B as a cathode. First and second electrodes 34A and 34B may be at the same or differing axial positions along the prosthetic valve.

Typically, circuitry 40 is configured to apply pacing to a heart of the patient and/or sense an intracardiac electrogram (EGM) of the heart using the one of first and second electrodes 34A and 34B as the anode and the other of first and second electrodes 34A and 34B as the cathode. Alternatively, one of first and second electrodes 34A and 34B may serve as an anode or a cathode, and another electrode 34 at a different axial location may serve as the other opposite-polarity electrode.

Reference is made to FIG. 8. In an application of the present invention, a method is provided that comprises:
delivering prosthetic atrioventricular valve 720 to native atrioventricular valve 754 of a heart of a patient in a constrained delivery configuration; and
transitioning prosthetic atrioventricular valve 720 to the expanded deployment configuration in which (a) the plurality of prosthetic leaflets 32 allow blood flow in a downstream direction and inhibit blood flow in an upstream direction, (b) first electrode 34A is in contact with one or more surfaces selected from the group consisting of: one or more downstream surfaces 702 of one or more respective leaflets 704 of native atrioventricular valve 754, and ventricular wall 706 in subvalvular space 708 of native atrioventricular valve 754.

For some of these applications:
prosthetic atrioventricular valve 720 further comprises the plurality of anchoring arms 756 coupled to frame 730,
first electrode 34A is coupled to one of anchoring arms 756, and
transitioning comprises transitioning prosthetic atrioventricular valve 720 to the expanded deployment configuration in which anchoring arms 756 extend in a radially outward and upstream direction, so as to engage one or more portions of the heart and thereby help hold prosthetic atrioventricular valve 720 in place upon deployment, the one or more portions selected from the group consisting of: chordae tendineae 758 of the heart, and downstream surfaces 702 of leaflets 704 of native atrioventricular valve 754.

This method may optionally be implemented in combination with any of the techniques described hereinabove with reference to FIGS. 7A-B and 8.

Reference is still made to FIG. 8. In an application of the present invention, a method is provided that comprises:
delivering a prosthetic atrioventricular valve 720 to native atrioventricular valve 754 of a heart of a patient in a constrained delivery configuration, prosthetic atrioventricular valve 754 including frame 730, a plurality of prosthetic leaflets 32 coupled to frame 730, a plurality of anchoring arms 756, and first and second electrodes 34A and 34B, which are coupled to first and second ones of the anchoring arms 756A and 756B, respectively; and
transitioning prosthetic atrioventricular valve 720 to an expanded deployment configuration in which (a) the plurality of prosthetic leaflets 32 allow blood flow in a downstream direction and inhibit blood flow in an upstream direction, and (b) anchoring arms 756 extend in a radially outward and upstream direction, so as to engage one or more portions of the heart and thereby help hold prosthetic atrioventricular valve 720 in place upon deployment, the one or more portions selected from the group consisting of: chordae tendineae 758 of the heart, and downstream surfaces 702 of leaflets 704 of native atrioventricular valve 754.

This method may optionally be implemented in combination with any of the techniques described hereinabove with reference to FIGS. 7A-B and 8.

Reference is now made to FIGS. 9A and 9B, which are schematic illustrations of two configurations of another prosthetic atrioventricular valve 820, in accordance with respective applications of the present invention. Other than as described hereinbelow, prosthetic atrioventricular valve 820 may be generally similar to prosthetic atrioventricular valve 720, described hereinabove with reference to FIGS. 7A-B and 8; prosthetic aortic valve 20, described hereinabove with reference to FIGS. 1A-3I; and/or prosthetic atrioventricular valve 520, described hereinabove with reference to FIG. 5, and may implement any of the features of any of these valves, mutatis mutandis. Prosthetic atrioventricular valve 820 may also optionally implement any of the features described hereinabove with reference to FIG. 4, mutatis mutandis, and/or described in PCT Publication WO 2025/041129 to Gross et al., with reference to FIGS. 4-8B thereof, mutatis mutandis. Like reference numerals refer to like elements.

Prosthetic atrioventricular valve 820 may, for example, be a prosthetic tricuspid valve or a prosthetic mitral valve. Optionally, prosthetic atrioventricular valve 820 implements any techniques known in the art for transcatheter prosthetic atrioventricular valves. For example, prosthetic atrioventricular valve 820 may implement techniques described in U.S. Pat. No. 11,246,704 to Hariton et al. and/or PCT Publication WO 2022/118316 to Albitov et al., and/or in US Patent Application Publication 2015/0328000 to Ratz et al., PCT Publication WO 2024/010739 to Garete et al., U.S. Pat. No. 10,973,628 to Levi, U.S. Pat. No. 10,299,927 to McLean et al., U.S. Pat. No. 10,828,153 to Noe et al., and/or U.S. Pat. No. 7,510,575 to Spenser et al., all of which are incorporated herein by reference.

Reference is made to FIG. 9A. For some applications, electrodes 34 are coupled to prosthetic atrioventricular valve 820 at or near (e.g., within 15 mm, such as within 8 mm) of an upstream end 846 of stent cells of the valve (the stent cells are present but cannot be seen in FIGS. 9A and 9B because they are covered by a sheet 802 that covers at least a portion of the stent cells.

Reference is made to FIG. 9B. In some applications, first and second electrodes 34A and 34B are coupled to first and second ones 856A and 856B of anchoring arms 856, respectively, of prosthetic atrioventricular valve 820. Anchoring arms 856 may be coupled to, or integral with, a frame of prosthetic atrioventricular valve 820. Optionally, additional electrodes 34, such as third electrode 34C, are coupled to additional ones of anchoring arms 856, respectively. The coupling of the electrodes to the anchoring arms may be performed as described hereinabove with reference to FIGS. 8B and 9, mutatis mutandis. Anchoring arms 856 may implement any of the features of anchoring arms 756 described hereinbelow with reference to FIGS. 8A-B and 9, mutatis mutandis. This electrode positioning may optionally be implemented with the electrode positioning described hereinabove with reference to FIG. 9A.

Reference is now made to FIG. 10, which is a schematic illustration of yet another prosthetic atrioventricular valve 920, in accordance with an application of the present invention. Other than as described hereinbelow, prosthetic atrioventricular valve 920 may be generally similar to prosthetic atrioventricular valve 820, described hereinabove with reference to FIGS. 9A-B; prosthetic atrioventricular valve 720, described hereinabove with reference to FIGS. 7A-B and 8; prosthetic aortic valve 20, described hereinabove with reference to FIGS. 1A-3I; and/or prosthetic atrioventricular valve 520, described hereinabove with reference to FIG. 5, and may implement any of the features of any of these valves, mutatis mutandis. Prosthetic atrioventricular valve 920 may also optionally implement any of the features described hereinabove with reference to FIG. 4, mutatis mutandis, and/or described in PCT Publication WO 2025/041129 to Gross et al., with reference to FIGS. 4-8B thereof, mutatis mutandis. Like reference numerals refer to like elements.

Prosthetic atrioventricular valve 920 may, for example, be a prosthetic tricuspid valve or a prosthetic mitral valve. Optionally, prosthetic atrioventricular valve 920 implements any techniques known in the art for transcatheter prosthetic atrioventricular valves. For example, prosthetic atrioventricular valve 920 may implement techniques described in U.S. Pat. No. 10,828,153 to Noe et al. and/or in PCT Publication WO 2022/118316 to Albitov et al., U.S. Pat. No. 11,246,704 to Hariton et al., US Patent Application Publication 2015/0328000 to Ratz et al., PCT Publication WO 2024/010739 to Garete et al., U.S. Pat. No. 10,973,628 to Levi, U.S. Pat. No. 10,299,927 to McLean et al., and/or U.S. Pat. No. 7,510,575 to Spenser et al., all of which are incorporated herein by reference.

For some applications, prosthetic atrioventricular valve 920 comprises:
an inner frame 922, which comprises inner interconnected stent struts 990A arranged so as to define inner interconnected stent cells 992A;
an outer frame 924, which comprises outer interconnected stent struts 990B arranged so as to define outer interconnected stent cells 992B, and which is coupled to inner frame 922 surrounding at least an axial portion 926 of inner frame 922 so that first and second frames 922 and 924 together define a central longitudinal axis when prosthetic atrioventricular valve 920 is in an expanded deployment configuration, such as shown in FIG. 10;
a plurality of prosthetic leaflets 932 coupled to inner frame 922 so as to allow blood flow in a downstream direction and inhibit blood flow in an upstream direction; and
electrodes 34, which include first and second electrodes 34A and 34B.

Typically, prosthetic atrioventricular valve 920 is configured such that, when prosthetic atrioventricular valve 920 is in the expanded deployment configuration, inner frame 922 applies an outwardly-directed radial force to assist with fixation of outer frame 924 to native anatomy of the patient, typically in the vicinity of the native atrioventricular valve.

For some applications, first electrode 34A is coupled to outer frame 924. Optionally, second electrode 34B is also coupled to outer frame 924. Optionally, additional electrodes 34, such as a third electrode 34C, are also coupled to outer frame 924. Optionally, one or more of electrodes 34 are coupled to outer frame 924 by being coupled to stent struts, such as interconnected stent struts 990B (optionally at peaks and/or junctions), or by being coupled to a skirt 938 (such that the electrodes are coupled to the outer frame via the skirt).

For some applications, electrodes 34 are coupled to prosthetic atrioventricular valve 920 at a downstream half, e.g., a downstream third, such as a downstream quarter of inner and outer interconnected stent cells 992A and 992B, taken collectively; for example, electrodes 34 may be coupled to prosthetic atrioventricular valve 920 within 2 cm of a downstream end 948 of outer interconnected stent cells 992B.

Alternatively or additionally, one or more of electrodes 34 are coupled to one or more elements of the valve other than outer frame 924, such as to inner frame 922. For some applications, inner and outer frames 922 and 924 are shaped such that when prosthetic atrioventricular valve 920 is in the expanded deployment configuration after deployment at a native atrioventricular valve, first and second electrodes 34A and 34B are disposed at a height of an annulus of the native cardiac valve.

For some applications, prosthetic atrioventricular valve 720 is configured, when in the expanded deployment configuration, to bring first electrode 34A (and, optionally, additional electrodes 34, such as second electrode 34B) into contact with one or more surfaces selected from the group consisting of: the one or more downstream surfaces 702 of the one or more respective leaflets 704 of native atrioventricular valve 754, and ventricular wall 706 in subvalvular space 708 of native atrioventricular valve 754 (labeled in FIG. 8). For example, outer frame 924 may be configured, when prosthetic atrioventricular valve 920 is in the expanded deployment configuration, to bring the one or more electrodes 34 into the contact with the one or more surfaces mentioned above; other portions of prosthetic atrioventricular valve 920 may also be configured to bring the one or more electrodes into this tissue contact.

The techniques described herein for prosthetic aortic valve 20 and the other prosthetic cardiac valves may be alternatively used, mutatis mutandis, for non-aortic prosthetic valves, such as prosthetic atrioventricular valves (prosthetic mitral valves or prosthetic tricuspid valves (sometimes referred to in the art as prosthetic orthotopic tricuspid valves), such as described hereinabove with reference to FIGS. 5, 7A-B, 8, 9A-B, and/or 10, or prosthetic pulmonary valves. In applications in which the techniques described herein are implemented in aortic, atrioventricular, or pulmonary valves, the prosthetic valves are typically implanted in and/or in the vicinity of the native annulus. The prosthetic valves described herein may also be implanted within vessels communicating with the heart, including a pulmonary artery (for replacing the function of a diseased pulmonary valve), or the superior vena cava or the inferior vena cava, or various other veins, arteries and vessels of a patient. In configurations in which the prosthetic valves are implanted in the superior vena cava or the inferior vena cava, the prosthetic valves replace the function of a diseased tricuspid valve; such prosthetic valves are known as prosthetic caval valves or heterotopic tricuspid valves, and the implantation procedure is sometimes referred to as caval valve implantation (CAVI). The prosthetic valves described herein may also be implanted within a previously implanted prosthetic valve (which can be a prosthetic surgical valve or a prosthetic transcatheter heart valve) in a valve-in-valve procedure.

In an embodiment, techniques and apparatus described in one or more of the following patents and/or applications, which are assigned to the assignee of the present application and are incorporated herein by reference, are combined with techniques and apparatus described herein:
U.S. Pat. No. 10,543,083 to Gross
European Patent Application Publication EP 3508113 A1 to Gross
U.S. Pat. No. 10,835,750 to Gross
U.S. Pat. No. 11,013,597 to Gross
PCT Publication WO 2021/140507 to Gross
PCT Publication WO 2021/224904 to Gross
U.S. Pat. No. 11,065,451 to Gross
U.S. Pat. No. 11,291,844 to Gross
PCT Publication WO 2022/149130 to Gross
U.S. patent application Ser. No. 18/452,216, filed Aug. 18, 2023, which issued as U.S. Pat. No. 11,975,203 to Gross et al.
U.S. patent application Ser. No. 18/452,229, filed Aug. 18, 2023, which issued as U.S. Pat. No. 11,931,255 to Gross et al.
U.S. patent application Ser. No. 18/607,638, filed Mar. 18, 2024, which published as US Patent Application Publication 2025/0058124 to Gross et al.
PCT Publication WO 2025/041129 to Gross et al.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. A prosthetic cardiac valve, which is configured to be delivered to a patient in a constrained delivery configuration, and which comprises:
a frame, which defines a central longitudinal axis when the prosthetic cardiac valve is in an expanded deployment configuration, and which comprises interconnected stent struts arranged so as to define interconnected stent cells that include upstream-most stent cells and non-upstream-most stent cells;
a plurality of prosthetic leaflets coupled to the frame so as to allow blood flow in a downstream direction and inhibit blood flow in an upstream direction; and
first and second electrodes, which are disposed at or near respective upstream peaks of first and second ones of the non-upstream-most stent cells, respectively.

2. The prosthetic cardiac valve according to claim 1, wherein the first and the second electrodes are disposed at a common axial position along the central longitudinal axis when the prosthetic cardiac valve is in the expanded deployment configuration.

3. The prosthetic cardiac valve according to claim 2, wherein the frame is shaped such that when the prosthetic cardiac valve is in the expanded deployment configuration after deployment at a native cardiac valve, the first and the second electrodes are disposed at a height of an annulus of the native cardiac valve.

4. The prosthetic cardiac valve according to claim 1, wherein the first and the second electrodes are disposed at different respective axial positions along the central longitudinal axis when the prosthetic cardiac valve is in the expanded deployment configuration.

5. The prosthetic cardiac valve according to claim 4, wherein the first and the second electrodes are disposed at a common angular location with respect to the central longitudinal axis when the prosthetic cardiac valve is in the expanded deployment configuration.

6. The prosthetic cardiac valve according to claim 1, further comprising a third electrode, which is disposed at or near an upstream peak of a third one of the non-upstream-most stent cells.

7. (canceled)

8. The prosthetic cardiac valve according to claim 1, wherein the first and the second non-upstream-most stent cells are located in an upstream half of the frame.

9. The prosthetic cardiac valve according to claim 1,
wherein the first and the second electrodes are disposed at respective axial positions along the central longitudinal axis when the prosthetic cardiac valve is in the expanded deployment configuration, the axial positions the same or differing from one another, and
wherein each of the axial positions is at a downstream-end distance from a downstream end of the stent cells and is at an upstream-end distance from an upstream end of the stent cells,
wherein the downstream-end distance equals at least 20% of a total axial length of the stent cells measured between the downstream end and the upstream end of the stent cells,
wherein the upstream-end distance equals at least 20% of the total axial length of the stent cells, and
wherein the downstream-end distance, the upstream-end distance, and the total axial length of the stent cells are measured parallel to the central longitudinal axis.

10-16. (canceled)

17. The prosthetic cardiac valve according to claim 1, wherein the frame further comprises one or more delivery-tool-coupling tabs, which extend axially beyond the stent cells.

18. The prosthetic cardiac valve according to claim 1, wherein the prosthetic cardiac valve further comprises:
circuitry, which is configured to use the first electrode as an anode and the second electrode as a cathode; and
first and second electrical leads, which electrically couple the first and the second electrodes, respectively, to the circuitry.

19. (canceled)

20. The prosthetic cardiac valve according to claim 18, wherein the circuitry is configured to apply pacing to a heart of the patient using the first electrode as the anode and the second electrode as the cathode.

21. The prosthetic cardiac valve according to claim 18, wherein the circuitry is configured to sense cardiac electrical activity of the patient using the first electrode as the anode and the second electrode as the cathode.

22. The prosthetic cardiac valve according to claim 1,
wherein the prosthetic cardiac valve further comprises an outer skirt,
wherein a portion of the stent cells is coupled to a radially inner surface of the outer skirt so that the outer skirt covers a first axial portion of the stent cells and does not cover a second axial portion of the stent cells, and an edge of the outer skirt is disposed around a circumference of the stent cells axially at a border between the first and the second axial portions of the stent cells,
wherein the first and the second electrodes are disposed at least partially on a radially outer surface of the outer skirt, within 8 mm of the edge of the outer skirt when the prosthetic cardiac valve is in the expanded deployment configuration.

23. The prosthetic cardiac valve according to claim 22, wherein the first and the second electrodes are disposed at least partially on the radially outer surface of the outer skirt, within 4 mm of the edge of the outer skirt when the prosthetic cardiac valve is in the expanded deployment configuration.

24. (canceled)

25. The prosthetic cardiac valve according to claim 22, wherein the first axial portion of the stent cells is downstream of the second axial portion of the stent cells.

26. (canceled)

27. The prosthetic cardiac valve according to claim 22, wherein the prosthetic cardiac valve further comprises circuitry and first and second electrical leads, which:
electrically couple the first and the second electrodes, respectively, to the circuitry, and
are coupled to interconnected stent struts of the second axial portion of the stent cells, and are not coupled to interconnected stent struts of the first axial portion of the stent cells.

28. The prosthetic cardiac valve according to claim 27, wherein the first and the second electrical leads run along respective portions of the interconnected stent struts to which the first and the second electrical leads are respectively coupled.

29. The prosthetic cardiac valve according to claim 27,
wherein the prosthetic cardiac valve further comprises an inner skirt,
wherein at least a portion of the stent cells is coupled to a radially outer surface of the inner skirt so that the inner skirt at least partially covers the second axial portion of the stent cells, and
wherein the first and the second electrical leads are coupled to at least a portion of the interconnected stent struts covered by the inner skirt.

30. The prosthetic cardiac valve according to claim 1, wherein the prosthetic cardiac valve is a prosthetic aortic valve.

31. The prosthetic cardiac valve according to claim 1, wherein the prosthetic cardiac valve is a prosthetic atrioventricular valve.

32-33. (canceled)

34. The prosthetic cardiac valve according to claim 1, wherein the prosthetic cardiac valve is a prosthetic pulmonary valve.

35. The prosthetic cardiac valve according to claim 1, wherein the prosthetic cardiac valve is a prosthetic caval valve.

36-155. (canceled)
